(19) United States
(12) Patent Application Publication
(10) Pub. No.: US 2017/0340740 A1
(43) Pub. Date: Nov. 30, 2017

(54) LONG-ACTING POLYMERIC DELIVERY SYSTEMS COMPRISING OLANZAPINE AND A 5-HT3 RECEPTOR ANTAGONIST

(71) Applicant: Heron Therapeutics, Inc., Redwood City, CA (US)

(72) Inventors: Thomas B. Ottoboni, Belmont, CA (US); Lee Ann Lynn Girotti, San Bruno, CA (US)

(21) Appl. No.: 15/604,435

(22) Filed: May 24, 2017

Related U.S. Application Data
(60) Provisional application No. 62/341,557, filed on May 25, 2016.

(51) Int. Cl. A61K 47/34; A61K 31/5513; A61K 31/439

(52) U.S. Cl. CPC A61K 47/34; A61K 31/439; A61K 31/5513

(57) ABSTRACT

Compositions comprised of olanzapine and a 5-HT3 receptor antagonist and a polyorthoester polymer are provided to provide extended release of the active agents. Also described are compositions comprising olanzapine and a polyorthoester. The compositions are effective for the prophylactic treatment or treatment of subjects at risk of or suffering from nausea and/or vomiting. The compositions are particularly useful for the prevention or treatment of acute, delayed, breakthrough or refractory chemotherapy induced nausea and vomiting (CINV). The CINV may result from, e.g., highly or moderately emetogenic cancer chemotherapy. The compositions are suitable for delivery via, e.g., intramuscular injection, intradermal injection, and subcutaneous injection.

### CROSS-REFERENCE TO RELATED APPLICATIONS

This application claims the benefit of U.S. Provisional Application No. 62/341,557, filed May 25, 2016, which is incorporated herein by reference in its entirety.

### TECHNICAL FIELD

The present disclosure is directed to compositions for delivery of anti-emetic agents to subjects in need thereof. In some embodiment, compositions disclosed herein provide delivery of one or more active agents over a period of up to about eight days. Exemplary compositions are formulated for the treatment or prophylactic treatment of nausea and vomiting which may be caused, for example, by chemotherapy.

### BACKGROUND

Nausea and vomiting may be caused by a variety of factors or situations. Certain drug treatments, such as cancer chemotherapy, antibiotics, and analgesics such as opioids can cause nausea and vomiting. Depending upon the chemotherapy agents or regimens given, up to 90% of patients may suffer from some form of chemotherapy-induced nausea and vomiting (CINV) in the absence of antiemetics. Symptoms from CINV are debilitating and can result in some patients refusing further courses of chemotherapy, with obviously unfavorable consequences in regard to progression of the cancer. Furthermore, if CINV cannot be controlled in an outpatient facility, patients may subsequently need treatment in an emergency room or require hospitalization. Postoperative nausea and vomiting (PONV) vomiting can also occur during the first 24 to 48 hours after surgery in inpatients.

Serotonin receptor antagonists (5-HT3 receptor antagonists), neurokinin-1 (NK1) receptor antagonists, and glucocorticoids have the highest therapeutic index for the prevention and/or treatment of CINV, and can be used alone or in combination (Smith et al., 2012, Ann Palliat Med, 1:115-120; Biomed Res Int, 2015, Article ID 495704). The mechanisms by which these agents reduce or prevent CINV are complex and diverse and the antiemetic most effective in treating or preventing CINV will vary based on whether, e.g., the CINV is acute, delayed, or breakthrough. Some patients are refractory to currently available antiemetogenic therapies. While great advances have been made over the past few decades for the treatment of acute emesis and CINV, effective treatment of delayed and breakthrough CINV can be challenging for many patients.

Olanzapine, an antipsychotic agent used to treat schizophrenia, has recently been shown to provide antiemetic efficacy. Olanzapine functions as an antagonist of many different receptors such as dopaminergic, serotonergic, adrenergic, histaminergic and muscarinic receptors (Brafford et al., 2014, J Adv Pract Oncol, 5:24-29). More recently, studies have shown olanzapine to be effective in the treatment of acute and delayed CINV (Navari, 2015, Biomed Res Int., vol. 2015, article ID 595894), including in patients refractory to other antiemetics (Srivastava et al., 2003, J Pain Symptom Manage, 25:578-582). Olanzapine has been shown to have efficacy in treating patients undergoing highly emetic chemotherapy (Babu et al., 2016, Chemother Res Pract, 2016:3439707; Wang et al., 2014, Asian Pac J Cancer Prev, 15:9587-9592) or moderately emetic chemotherapy (Navari et al., Support Care Cancer, 2007, 15:1285-1291; Navari et al., 2006, J Clin Oncol, Abst. Vol. 24, No. 18S).

As the use of olanzapine for treatment of CINV is further studied and developed, there is a need to identify optimal compositions and modes of administration in combination with other antiemetics which are most effective in prophylactically treating or treating nausea and vomiting resulting from highly and moderately emetogenic chemotherapy. One approach for optimizing treatment of nausea and vomiting caused by CINV is the utilization of sustained or extended release systems provided by the present compositions.

### BRIEF SUMMARY

In one aspect, a composition comprising olanzapine and a delivery vehicle is provided.

In one aspect, a composition comprising a 5-HT3 receptor antagonist, olanzapine and a delivery vehicle is provided.

In some embodiments, the composition is an aqueous based solution.

In other embodiments, the delivery vehicle is a sustained release delivery vehicle. In yet other embodiments, the sustained release delivery vehicle is a liposome selected from the group consisting of small unilamellar vesicles (SUV), large unilamellar vesicles (LUV), multi-lamellar vesicles (MLV) and multivesicular liposomes (MVL).

In some embodiments, the sustained-release delivery vehicle is a polymeric composition, a liposomal composition, a microsphere composition, a non-polymeric composition or an implantable device. In other embodiments, the sustained-release vehicle is a liposomal composition and the olanzapine and/or the 5-HT3 receptor antagonist are entrapped in an aqueous space of a liposome or in a lipid layer of the liposome. In still other embodiments, the sustained release vehicle is a microsphere comprising a bioerodible or biodegradable polymer. In yet other embodiments, the olanzapine and/or the 5-HT3 receptor antagonist are entrapped in the microsphere. In still other embodiments, the implantable device is an osmotic pump with a reservoir comprising the olanzapine and the 5-HT3 receptor antagonist.

In some embodiments, the sustained release delivery vehicle is not a microsphere composition.

In some embodiments, the sustained release delivery vehicle is not a liposomal composition.

In some embodiments, the sustained release delivery vehicle is not a non-polymeric composition.

In some embodiments, the sustained release delivery vehicle is not an implantable device.

In some embodiments, the composition is injectable. In other embodiments, the composition is suitable for administration as an intramuscular, intravenous, transdermal, or subcutaneous injection. In other embodiments, the composition is suitable to topical administration.

In some embodiments, the composition has a viscosity of less than 10,000 mPa-s, less than 5,000 mPa-s or less than 2500 mPa-s when viscosity is measured at 37° C. using a cone and plate viscometer. In other embodiments, the composition has a viscosity of less than 10,000 mPa-s, less than 5,000 mPa-s, or less than 2,500 mPa-s when viscosity is measured at 25° C. using a cone and plate viscometer.

In other embodiments, the sustained release delivery vehicle is a non-polymeric composition comprising sucrose acetate isobutyrate.

In some embodiments, the sustained release delivery vehicle is a polymeric composition in the form of a semi-solid polymer formulation comprising a polymer, the olanzapine and the 5-HT3 receptor antagonist. In other embodiments, the polymer formulation forms an implant or depot in situ.

In some embodiments, the polymer is a bioerodible or biodegradable polymer.

In still other embodiments, the polymer is selected from the group consisting of polylactides, polyglycolides, poly(lactic-co-glycolic acid) copolymers, polycaprolactones, poly-3-hydroxybutyrates, and polyorthoesters.

In some embodiments, the sustained release delivery vehicle comprises a polyorthoester, the 5-HT3 receptor antagonist and the olanzapine.

In some embodiments, the 5-HT3 receptor antagonist is selected from the group consisting of granisetron, tropisetron, ondansetron, palonosetron, and dolasetron. In specific embodiments, the 5-HT3 receptor antagonist is granisetron.

In a particular embodiment related to any one or more of the foregoing embodiments, the sustained release delivery vehicle comprises olanzapine and granisetron.

In some embodiments, the biodegradable or bioerodible polymeric formulation comprises a polymer selected from the group consisting of polylactide, polyglycolide, a poly(lactic-co-glycolic acid) copolymer, polycaprolactone, poly-3-hydroxybutyrate, or a polyorthoester. In a particular embodiment, the polymer is a polyorthoester.

In some particular embodiments, the polyorthoester is selected from the polyorthoesters represented by Formulas I, II, III and IV as set forth herein below.

In yet a particular embodiment related to the foregoing, the polyorthoester is represented by Formula I as set forth herein.

In some embodiments, the polyorthoester is represented by the structure shown as Formula

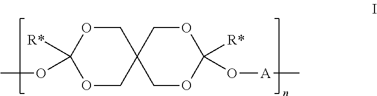

where: R* is a methyl, ethyl, propyl or butyl, n is the number of repeating units and is an integer ranging from 5 to 400, and A in each subunit is R¹ or R³.

In some embodiments directed to Formula I, R* is ethyl.

In yet some additional embodiments directed to Formula I, A corresponds to R¹, where R¹ is

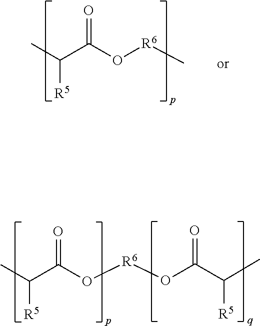

where p and q are each independently integers ranging from about 1 to 20, each R⁵ is independently hydrogen or C₁₋₄ alkyl; and R⁶ is:

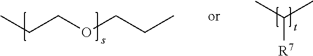

where s is an integer from 0 to 10; t is an integer from 2 to 30; and R⁷ is hydrogen or C₁₋₄ alkyl.

In some other embodiments related to Formula I, R⁷ is C1, C2, C3, or C4 alkyl. In some particular embodiments, R⁷ is H.

In yet still other embodiments, the R¹ subunits are α-hydroxy acid-containing subunits.

In yet other embodiments, p and q are each independently selected from 1, 2, 3, 4, 5, 6, 7, 8, 9, 10, 11, 12, 13, 14, 15, 16, 17, 18, 19, and 20.

In yet another embodiment, R⁵ is independently hydrogen, or C1, C2, C3, or C4 alkyl.

In some embodiments, A corresponds to R³, where R³ is:

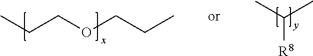

and x is an integer ranging from 1 to 100. In another embodiment, x is selected from 0, 1, 2, 3, 4, and 5; y is an integer in a range from 2 to 30; and R⁸ is hydrogen or C₁₋₄ alkyl. In still another embodiment, R⁸ is a C1, C2, C3 or C4 alkyl. In another embodiment, R⁸ is H.

In some embodiments, the polyorthoester is one of Formula I, II, III or IV, and in particular of Formula I, in which A is R¹ or R³, where R¹ is

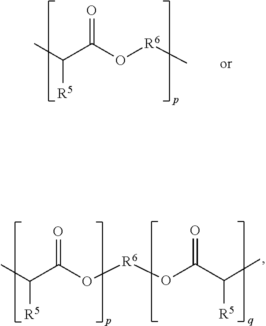

where p and q are each independently selected from 1, 2, 3, 4, 5, 6, 7, 8, 9, 10, 11, 12, 13, 14, 15, 16, 17, 18, 19, and 20 in any repeating unit, where the average number of p or the average number of the sum of p and q (p+q) is between about 1 and 7; x and s are each independently an integer ranging from 0 to 10; and t and y are each independently an integer ranging from 2 to 30. In yet additional embodiments, the sum of p and q is 1, 2, 3, 4, 5, 6 or 7 in any repeating unit of R¹. In yet some further embodiments, R⁵ is H.

In yet further embodiments, A is R¹ or R³, where R¹ is

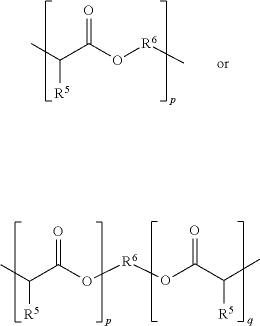

and p and q are each independently integers ranging from about 1 and 20, about 1 and 15, or about 1 and 10 in any repeating unit of R¹, where the average number of p or the average number of the sum of p and q (i.e., p+q) is between about 1 and 7. In another one or more embodiments, x and s each independently range from 0 to about 7 or from 1 to about 5. In still another embodiment, t and y each independently range from 2 to 10.

In one embodiment, R⁵ is hydrogen or methyl.

In one embodiment, s and x are each independently selected from 1, 2, 3, 4, 5, 6, 7 and 8. In some particular embodiments, s is 2. In still yet further embodiments, x is 2.

In one embodiment, the polyorthoester comprises alternating residues of 3,9-diethyl-3,9-2,4,8,10-tetraoxaspiro[5.5]undecane-3,9-diyl and A:

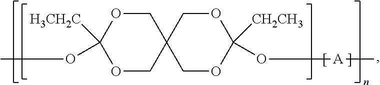

where A is as described above.

In yet one or more additional embodiments, the sustained release delivery vehicle comprising the polyorthoester, the olanzapine and the 5-HT3 receptor antagonist, further comprises a solvent. In some embodiments, the solvent is protic or aprotic in nature. In other embodiments, the solvent is dimethyl sulfoxide (DMSO).

In some embodiments, the combination of the polyorthoester and the solvent in the delivery vehicle is present in an amount ranging from about 85 to 98 wt %, 90 to 95 wt %, 93 to 95 wt %, or 93 to 97 wt %.

In some embodiments, the polyorthoester is present in the delivery vehicle in an amount ranging from about 65 to 95 wt %, 70 to 90 wt %, 70 to 85 wt %, 70 to 80 wt %, 75 to 90 wt %, 75 to 85 wt %, 80 to 90 wt %, or 85 to 90 wt %, or in an amount about 70 wt %, 73 wt %, 75 wt %, 78 wt %, 80 wt %, 85 wt % or 90 wt %.

In some embodiments, the solvent is present in the delivery vehicle in an amount ranging from about 5 to 30 wt %, 10 to 25 wt %, 10 to 23 wt %, 15 to 25 wt %, 15 to 20 wt %, 16 to 18 wt %, 20 to 25 wt %, or in an amount about 10 wt %, 15 wt %, 17 wt %, 20 wt %, 23 wt % or 25 wt %.

In yet one or more further embodiments, the active agents are solubilized in the sustained release delivery vehicle.

In some embodiments, the composition comprises a polyorthoester according to any one of the foregoing embodiments, olanzapine, granisetron, and DMSO.

In some embodiments related to the composition, the olanzapine is present the delivery vehicle in an amount ranging from about 1 to 8 wt %, 1 to 6 wt %, 1 to 5 wt %, 0.5 to 5 wt %, 0.5 to 4 wt %, 0.5 to 3 wt %, 1 to 4 wt %, 1 to 3 wt %, or 1.5 to 2.5 wt % of the delivery vehicle. In other embodiments, olanzapine is present the delivery vehicle in an amount of about 0.5 wt %, 0.6 wt %, 0.7 wt %, 0.8 wt %, 0.9 wt %, 1 wt %, 1.25 wt %, 1.5 wt %, 1.75 wt %, 2 wt %, 2.25 wt %, 2.5 wt %, 2.75 wt %, 3 wt %, 3.25 wt %, 3.5 wt %, 3.75 wt %, 4 wt %, 4.25 wt %, 4.5 wt %, 4.75 wt %, 5 wt %, 5.25 wt %, 5.5 wt %, 5.75 wt %, 6 wt %, 6.25 wt %, 6.5 wt %, 6.75 wt %, 7 wt %, 7.25 wt %, 7.5 wt %, 7.75 wt %, 8 wt %, 8.25 wt %, or 8.5 wt %.

In some embodiments related to the composition, the 5-HT3 receptor antagonist is present in the delivery vehicle in an amount ranging from about 1 to 10 wt %, 1 to 8 wt %, 1 to 6 wt %, 1 to 5 wt %, 0.5 to 5 wt %, 0.5 to 4 wt %, 0.5 to 3 wt %, 1 to 4 wt %, 1 to 3 wt %, or 1.5 to 2.5 wt % of the delivery vehicle. In other embodiments, the 5-HT3 receptor antagonist is present in the delivery vehicle at about 0.5 wt %, 0.6 wt %, 0.7 wt %, 0.8 wt %, 0.9 wt %, 1 wt %, 1.25 wt %, 1.5 wt %, 1.75 wt %, 2 wt %, 2.25 wt %, 2.5 wt %, 2.75 wt %, 3 wt %, 3.25 wt %, 3.5 wt %, 3.75 wt %, 4 wt %, 4.25 wt %, 4.5 wt %, 4.75 wt %, 5 wt %, 5.25 wt %, 5.5 wt %, 5.75 wt %, 6 wt %, 6.25 wt %, 6.5 wt %, 6.75 wt %, 7 wt %, 7.25 wt %, 7.5 wt %, 7.75 wt %, 8 wt %, 8.25 wt %, or 8.5 wt %. In a particular embodiment, the 5-HT3 receptor antagonist is granisetron.

In some embodiments related to the composition, the olanzapine and the 5-HT3 receptor antagonist are present in the delivery vehicle in an amount ranging from about 0.5 to 8 wt % and 1 to 10 wt %, respectively; 0.5 to 5 wt % and 1 to 10 wt %, respectively; 1 to 6 wt % and 1 to 8 wt %, respectively; 1 to 5 wt % and 2 to 6 wt %, respectively; and 1 to 3 wt % and 2 to 4 wt %, respectively. In a particular embodiment, the 5-HT3 receptor antagonist is granisetron.

In some embodiments related to the composition, the olanzapine is present in the delivery vehicle in an amount ranging from about 5 to 30 mg, 5 to 25 mg, 5 to 20 mg, 5 to 15 mg, 6 to 14 mg, 7 to 13 mg, 8 to 12 mg or about 1 mg, 1.25 mg, 1.5 mg, 1.75 mg, 2 mg, 2.25 mg, 2.5 mg, 2.75 mg, 3 mg, 3.25 mg, 3.5 mg, 3.75 mg, 4 mg, 4.25 mg, 4.5 mg, 4.75 mg, 5 mg, 5.25 mg, 5.5 mg, 5.75 mg, 6 mg, 6.25 mg, 6.5 mg, 6.75 mg, 7 mg, 7.25 mg, 7.5 mg, 7.75 mg, 8 mg, 8.25 mg, 8.5 mg, 8.75 mg, 9 mg, 9.25 mg, 9.5 mg, 9.75 mg, 10 mg, 10.25 mg, 10.5 mg, 10.75 mg, 11 mg, 11.25 mg, 11.5 mg, 11.75 mg, 12 mg, 12.25 mg, 12.5 mg, 12.75 mg, 13 mg, 13.25 mg, 13.5 mg, 13.75 mg, 14 mg, 14.25 mg, 14.5 mg, 14.75 mg, 15 mg, 15.25 mg, 15.5 mg, 15.75 mg, 16 mg, 16.25 mg, 16.5 mg, 16.75 mg, 17 mg, 17.25 mg, 17.5 mg, 17.75 mg, 18 mg, 18.25 mg, 18.5 mg, 18.75 mg, 19 mg, 19.25 mg, 19.5 mg, 19.75 mg, 20 mg, 20.25 mg, 20.5 mg, 20.75 mg, 21 mg, 21.25 mg, 21.5 mg, 21.75 mg, 22 mg, 22.25 mg, 22.5 mg, 22.75 mg, 23 mg, 23.25 mg, 23.5 mg, 23.75 mg, 24 mg, 24.25 mg, 24.5 mg, 24.75 mg, 25 mg, 25.25 mg, 25.5 mg, 25.75 mg, 26 mg, 26.25 mg, 26.5 mg, 26.75 mg, 27 mg, 27.25 mg, 27.5 mg, 27.75 mg, 28 mg, 28.25 mg, 28.5 mg, 28.75 mg, 29 mg, 29.25 mg, 29.5 mg, 29.75 mg, or 30 mg.

In some embodiments related to the composition, the 5-HT3 receptor antagonist is present in the delivery vehicle in an amount ranging from about 5 to 25 mg, 7 to 23 mg, 9 to 22 mg, 10 to 20 mg, 5 to 15 mg, 5 to 12 mg, 7 to 18 mg, or 8 to 12 mg or about 1 mg, 1.25 mg, 1.5 mg, 1.75 mg, 2 mg, 2.25 mg, 2.5 mg, 2.75 mg, 3 mg, 3.25 mg, 3.5 mg, 3.75 mg, 4 mg, 4.25 mg, 4.5 mg, 4.75 mg, 5 mg, 5.25 mg, 5.5 mg, 5.75 mg, 6 mg, 6.25 mg, 6.5 mg, 6.75 mg, 7 mg, 7.25 mg, 7.5 mg, 7.75 mg, 8 mg, 8.25 mg, 8.5 mg, 8.75 mg, 9 mg, 9.25 mg, 9.5 mg, 9.75 mg, 10 mg, 10.25 mg, 10.5 mg, 10.75 mg, 11 mg, 11.25 mg, 11.5 mg, 11.75 mg, 12 mg, 12.25 mg, 12.5 mg, 12.75 mg, 13 mg, 13.25 mg, 13.5 mg, 13.75 mg, 14 mg, 14.25 mg, 14.5 mg, 14.75 mg, 15 mg, 15.25 mg, 15.5 mg, 15.75 mg, 16 mg, 16.25 mg, 16.5 mg, 16.75 mg, 17 mg, 17.25 mg, 17.5 mg, 17.75 mg, 18 mg, 18.25 mg, 18.5 mg, 18.75 mg, 19 mg, 19.25 mg, 19.5 mg, 19.75 mg, 20 mg, 20.25 mg, 20.5 mg, 20.75 mg, 21 mg, 21.25 mg, 21.5 mg, 21.75 mg, 22 mg, 22.25 mg, 22.5 mg, 22.75 mg, 23 mg, 23.25 mg, 23.5 mg, 23.75 mg, 24 mg, 24.25 mg, 24.5 mg, 24.75 mg, or 25 mg. In a particular embodiment, the 5-HT3 receptor antagonist is granisetron.

In some embodiments related to the composition, the olanzapine and the 5-HT3 receptor antagonist are present in the delivery vehicle in an amount ranging from about 5 to 15 mg and 5 to 25 mg, respectively; 5 to 10 mg and 7 to 20 mg, respectively; 8 to 12 mg and 9 to 14 mg, respectively. In a particular embodiment, the 5-HT3 receptor antagonist is granisetron.

In some embodiments, the olanzapine is released from the delivery vehicle over a period ranging from about 12 to 120 hours, about 24 to 120 hours, about 48 to 120 hours, about 72 to 120 hours, about 78 to 120 hours, about 84 to 120 hours, about 90 to 120 hours, about 96 to 120 hours, about 102 to 120 hours, about 108 to 120 hours, about 116 to 120 hours, about 48 to 96 hours, about 72 to 96 hours, about 78 to 96 hours, about 84 to 96 hours, about 72 to 132 hours, about 96 hours to 132 hours, about 108 hours to 132 hours, about 108 to 144 hours, about 108 to 168 hours, or about 108 to 192 hours.

In some embodiments, the olanzapine and the 5-HT3 receptor antagonist are released from the delivery vehicle over a period ranging from about 12 to 120 hours, about 24 to 120 hours, about 48 to 120 hours, about 72 to 120 hours, about 78 to 120 hours, about 84 to 120 hours, about 90 to 120 hours, about 96 to 120 hours, about 102 to 120 hours, about 108 to 120 hours, about 116 to 120 hours, about 48 to 96 hours, about 72 to 96 hours, about 78 to 96 hours, about 84 to 96 hours, about 72 to 132 hours, about 96 hours to 132 hours, about 108 hours to 132 hours, about 96 hours to 132 hours, about 108 hours to 132 hours, about 108 to 144 hours, about 108 to 168 hours, or about 108 to 192 hours. In other embodiments, the 5-HT3 receptor antagonist is granisetron.

In one aspect, a method for treating a subject experiencing nausea and vomiting is provided, comprising administering to the subject a sustained release drug delivery vehicle related to any one or more of the foregoing embodiments comprising a bioerodible polymer and olanzapine.

In one aspect, a method for treating a subject experiencing nausea and vomiting is provided, comprising administering to the subject a sustained release drug delivery vehicle related to any one or more of the foregoing embodiments comprising a bioerodible polymer, a 5-HT3 receptor antagonist, and olanzapine. In some embodiments, the 5-HT3 receptor antagonist is selected from the group consisting of granisetron, tropisetron, ondansetron, palonosetron, and dolasetron. In particular embodiments, the 5-HT3 receptor antagonist is granisetron.

In some embodiments, a method for preventing nausea and/or vomiting associated with initial and repeat courses of highly emetogenic cancer therapy is provided. In other embodiments, the highly emetogenic cancer therapy is high-dose cisplatin.

In some embodiments, a method for preventing nausea and/or vomiting associated with initial and repeat courses of moderately emetogenic cancer therapy is provided.

In some embodiments, the subject is also treated with a glucocorticoid. In a particular embodiment, the glucocorticoid is dexamethasone.

In some embodiments, the subject is undergoing chemotherapy. In other embodiments, the patient is undergoing highly emetogenic chemotherapy (HEC). In still other embodiments, the patient is undergoing moderately emetogenic chemotherapy (MEC).

In some embodiments, the subject is suffering from acute or delayed onset chemotherapy-induced nausea and vomiting (CINV).

In some embodiments, the subject is suffering from breakthrough CINV.

In some embodiments, the subject is suffering from refractory CINV.

In some embodiments, the subject is suffering from post-operative nausea and vomiting (PONV).

In some embodiments, a method for preventing postoperative nausea and vomiting is provided.

In some embodiments related to any one or more embodiments as provided herein, the treatment method comprises administering to the patient a single dose of the semi-solid drug delivery vehicle comprising from about 1 to 20 mg of olanzapine during one cycle of chemotherapy. In a particular embodiment related to the foregoing, the single dose of the semi-solid drug delivery vehicle comprises about 3.5 to 8 mg of olanzapine.

In some embodiments related to any one or more embodiments as provided herein, the treatment method comprises administering to the patient a single dose of the semi-solid drug delivery vehicle comprising from 1 to 20 mg of olanzapine during one cycle of chemotherapy. In a particular embodiment related to the foregoing, the single dose of the semi-solid drug delivery vehicle comprises 3.5 to 8 mg of olanzapine.

In some embodiments related to any one or more embodiments as provided herein, the treatment method comprises administering to the patient a single dose of the semi-solid drug delivery vehicle comprising from 1 to 25 mg of granisetron and from 1 to 20 mg of olanzapine during one cycle of chemotherapy. In a particular embodiment related to the foregoing, the single dose of the semi-solid drug delivery vehicle comprises 5 to 10 mg of granisetron and 3.5 to 8 mg of olanzapine.

In some embodiments related to the foregoing, the single dose is administered prior to commencement of chemotherapy; in alternative embodiments, the single dose is administered post-chemotherapy. In other embodiments, the single dose is administered immediately after the completion of chemotherapy.

In some embodiments related to the foregoing, the single dose is administered prior to commencement of surgery; in other embodiments, the single dose is administered intrasurgically; in still other embodiments, the single dose is administered post-surgery.

In some embodiments, the treatment method which comprises administration of a dose comprising olanzapine and granisetron is effective to provide a measurable prevention or reduction of nausea and vomiting when compared to previous treatment with a 5-HT3 receptor antagonist other than granisetron. In other embodiments, the treatment method is effective to provide a measurable prevention or reduction of acute or delayed nausea and vomiting when compared to previous treatment with granisetron in the absence of olanzapine.

In some embodiments, the method is effective to result in a complete absence of an emetic episode in the acute phase of chemotherapy. In other embodiments, the method is effective to result in a complete absence of an emetic episode in the delayed phase of chemotherapy. In further embodiments, the method is effective to result in a complete absence of an emetic episode in both the acute and delayed phase of chemotherapy.

In some embodiments, the administering is continued over one or more repeated cycles of chemotherapy.

In some embodiments, the administration of the composition to the subject comprising olanzapine provides the subject with relief from nausea and/or vomiting for a duration of about 12 to 120 hours, about 24 to 120 hours, about 48 to 120 hours, about 72 to 120 hours, about 78 to 120 hours, about 84 to 120 hours, about 90 to 120 hours, about 96 to 120 hours, about 102 to 120 hours, about 108 to 120 hours, about 116 to 120 hours, about 48 to 96 hours, about 72 to 96 hours, about 78 to 96 hours, about 84 to 96 hours, about 72 to 132 hours, about 96 hours to 132 hours, or about 108 hours to 132 hours after administration. In other embodiments, the administration of the composition to the subject comprising olanzapine provides the subject with relief from nausea or nausea and vomiting for a duration of about 3 to 7 days, 3 to 6 days, 3 to 5 days, 4 to 5 days, 4 to 6 days, 4 to 7 days, 4 to 8 days, 5 to 8 days, 5 to 6 days, 1 to 3 days, or 2 to 4 days.

In some embodiments related to the foregoing, the administration of the composition to the subject comprising the 5-HT3 receptor antagonist and olanzapine provides the subject with relief from nausea and/or vomiting for a duration of about 12 to 120 hours, about 24 to 120 hours, about 48 to 120 hours, about 72 to 120 hours, about 78 to 120 hours, about 84 to 120 hours, about 90 to 120 hours, about 96 to 120 hours, about 102 to 120 hours, about 108 to 120 hours, about 116 to 120 hours, about 48 to 96 hours, about 72 to 96 hours, about 78 to 96 hours, about 84 to 96 hours, about 72 to 132 hours, about 96 hours to 132 hours, or about 108 hours to 132 hours after administration. In other embodiments related to the foregoing, the administration of the composition to the subject comprising the 5-HT3 receptor antagonist and olanzapine provides the subject with relief from nausea or nausea and vomiting for a duration of about 3 to 7 days, 3 to 6 days, 3 to 5 days, 4 to 5 days, 4 to 6 days, 4 to 7 days, 4 to 8 days, 5 to 8 days, 5 to 6 days, 1 to 3 days, or 2 to 4 days. In a particular embodiment, the 5-HT3 receptor antagonist is granisetron.

In one embodiment, the composition is administered by intravenous, subcutaneous, intradermal or intramuscular injection.

In another aspect, a method of administering a therapeutically active agent is provided. The method comprises dispensing from a needle a delivery system or a composition as described herein comprising a polyorthoester according to any one of the foregoing embodiments and the therapeutically active agents dispersed or solubilized in the single phase, wherein the solvent is selected to achieve a controlled release of the active agent or active agents from the composition according to a predetermined release profile, and wherein the active agent is or active agents are released from the delivery system or composition over a period ranging from about 12 to 120 hours, about 24 to 120 hours, about 48 to 120 hours, about 72 to 120 hours, about 78 to 120 hours, about 84 to 120 hours, about 90 to 120 hours, about 96 to 120 hours, about 102 to 120 hours, about 108 to 120 hours, about 116 to 120 hours, about 48 to 96 hours, about 72 to 96 hours, about 78 to 96 hours, about 84 to 96 hours, about 72 to 132 hours, about 96 hours to 132 hours, or about 108 hours to 132 hours after the administering. In one particular embodiment, the therapeutically active agents is olanzapine. In another particular embodiment, the therapeutically active agents are olanzapine and a 5-HT3 receptor antagonist. In some embodiments, the 5-HT3 receptor antagonist is granisetron. In a particular embodiment, the solvent is DMSO.

Additional embodiments of the present systems, compositions and methods will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present invention. Additional aspects and advantages of the present invention are set forth in the following description and claims, particularly when considered in conjunction with the accompanying examples and drawings.

### BRIEF DESCRIPTION OF THE FIGURES

FIG. 1 is a graph of the percent of olanzapine released in vitro as a function of time, in days, from delivery vehicle.

FIG. 2 is a graph of the percent of granisetron released as a function of time, in days, from a delivery vehicle in vitro.

FIG. 3 is a graph of plasma concentration of granisetron (squares) and olanzapine (diamonds), in ng/mL, as a function of time, in hours, for a delivery system comprising a polyorthoester, an aprotic solvent, granisetron and olanzapine.

### DETAILED DESCRIPTION

### I. Definitions

As used in this specification, the singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

Where a range of values is provided, it is intended that each intervening value between the upper and lower limit of that range and any other stated or intervening value in that stated range is encompassed within the disclosure. For example, if a range of 10 to 20 weight percent (wt %) is stated, it is intended that 11, 12, 13, 14, 15, 16, 17, 18, and 19 wt % are also explicitly disclosed, as well as the range of values greater than or equal to 10 wt % up to about 20 wt % and the range of values less than or equal to 20 wt % down to about 10 wt %.

“Bioerodible,” “bioerodibility” and “biodegradable,” which are used interchangeably herein, refer to the degradation, disassembly or digestion of a polymer by action of a biological environment, including the action of living organisms and most notably at physiological pH and temperature. As an example, a principal mechanism for bioerosion of a polyorthoester is hydrolysis of linkages between and within the units of the polyorthoester.

A “polymer susceptible to hydrolysis” such as a polyorthoester refers to a polymer that is capable of degradation, disassembly or digestion via reaction with water molecules. Such a polymer contains hydrolyzable groups in the polymer. Examples of polymers susceptible to hydrolysis may include, but are not limited to, polymers described herein, and those described in U.S. Pat. Nos. 4,079,038, 4,093,709, 4,131,648, 4,138,344, 4,180,646, 4,304,767, 4,957,998, 4,946,931, 5,968,543, 6,613,335, and 8,252,304, and U.S. Patent Publication No. 2007/0265329, which are incorporated herein by reference in its entirety.

“Molecular mass” in the context of a polymer such as a polyorthoester, refers to the nominal average molecular mass of a polymer, typically determined by size exclusion chromatography, light scattering techniques, or velocity. Molecular weight can be expressed as either a number-average molecular weight or a weight-average molecular weight. Unless otherwise indicated, all references to molecular weight herein refer to the weight-average molecular weight. Both molecular weight determinations, number-average and weight-average, can be measured using gel permeation chromatographic or other liquid chromatographic techniques. Other methods for measuring molecular weight values can also be used, such as the measurement of colligative properties (e.g., freezing-point depression, boiling-point elevation, or osmotic pressure) to determine number-average molecular weight or the use of light scattering techniques, ultracentrifugation or viscometry to determine weight-average molecular weight. The polymers of the invention are typically polydisperse (i.e., number-average molecular weight and weight-average molecular weight of the polymers are not equal), possessing low polydispersity values such as less than about 3.0, less than about 2.75, less than about 2.25, less than about 1.5, and less than about 1.03.

“Semi-solid” denotes the mechano-physical state of a material that is flowable under moderate stress. More specifically, a semi-solid material will generally have a viscosity between about 1,000 and 3,000,000 mPa-s at 37° C., especially between about 1,000 and 50,000 mPa-s at 37° C.

An “active agent” or “active ingredient” refers to any compound or mixture of compounds which produces a beneficial or useful result. Generally, “active agent” or “drug” refers to any organic or inorganic compound or substance having bioactivity and adapted or used for therapeutic purposes. As used herein, reference to a drug, as well as reference to other chemical compounds herein, is meant to include the compound in any of its pharmaceutically acceptable forms, including isomers such as diastereomers and enantiomers, salts, solvates, and polymorphs, particular crystalline forms, as well as racemic mixtures and pure isomers of the compounds described herein, where applicable. Active agents are distinguishable from such components as vehicles, carriers, diluents, lubricants, binders and other formulating aids, and encapsulating or otherwise protective components. Examples of active agents are pharmaceutical, agricultural or cosmetic agents.

A “small molecule” is a molecule, typically a drug, having a molecular weight of less than about 900 daltons.

“Pharmaceutically acceptable salt” denotes a salt form of a drug having at least one group suitable for salt formation that causes no significant adverse toxicological effects to the patient. Pharmaceutically acceptable salts include salts prepared by reaction with an inorganic acid, an organic acid, a basic amino acid, or an acidic amino acid, depending upon the nature of the functional group(s) in the drug. Suitable pharmaceutically acceptable salts include acid addition salts which may, for example, be formed by mixing a solution of a basic drug with a solution of an acid capable of forming a pharmaceutically acceptable salt form of the basic drug, such as hydrochloric acid, iodic acid, fumaric acid, maleic acid, succinic acid, acetic acid, citric acid, tartaric acid, carbonic acid, phosphoric acid, sulfuric acid and the like. Typical anions for basic drugs, when in protonated form, include chloride, sulfate, bromide, mesylate, maleate, citrate and phosphate. Suitable pharmaceutically acceptable salt forms are found in, e.g., Handbook of Pharmaceutical Salts: Properties, Selection and Use, Weinheim/Zürich:Wiley-VCH/VHCA, 2002; P. H. Stahl and C. G. Wermuth, Eds.

“Polyorthoester-compatible” refers to, in one particular aspect of the properties of the polyorthoester, the properties of an excipient which, when mixed with the polyorthoester, forms a single phase and does not cause any chemical changes to the polyorthoester.

A “therapeutically effective amount” means the amount that, when administered to a human or an animal for treatment of a disease or condition, is sufficient to effect treatment for that disease or condition.

“Treating” or “treatment” of a disease or condition includes preventing the disease or condition from occurring in a human or an animal that may be predisposed to the disease or condition but does not yet experience or exhibit symptoms of the disease or condition (prophylactic treatment), inhibiting the disease or condition (slowing or arresting its development), providing relief from the symptoms or side-effects of the disease or condition (including palliative treatment), and relieving the disease or condition (causing regression of the disease or condition).

As used herein, “synergistic” when used in relation to the combination refers to a combination that allows a lower amount of a first antiemetic agent (e.g., a 5-HT3 receptor antagonist such as granisetron) and, in some embodiments, also a lower amount of a second agent (e.g., olanzapine), than would be required to achieve a given level of relief from nausea and/or nausea and vomiting if the 5-HT3 receptor antagonist were administered alone. The synergistic combination may allow a lower amount of 5-HT3 receptor antagonist and olanzapine to be administered in a single dose to provide a given level of relief from nausea and/or nausea and vomiting than if the 5-HT3 receptor antagonist or olanzapine were administered alone thereby providing a greater than additive relief in combination. In some instances, the lower amount of the 5-HT3 receptor antagonist and olanzapine is a sub-therapeutic amount in which one or both of the components of the combination are administered at a dosage normally considered not to provide relief from nausea and/or nausea and vomiting.

Alternatively, the term “synergistic” when used in relation to the combination refers to a combination that extends the duration or degree of the relief from CINV, PONV or other disorders involving nausea and vomiting beyond the duration observed when either the olanzapine or the 5-HT3 receptor antagonist is administered alone. In this instance, the amount of olanzapine and/or the 5-HT3 receptor antagonist may be the same as the amount normally provided in a single dose to achieve relief from nausea and vomiting, thereby allowing a lower amount of olanzapine and the 5-HT3 receptor antagonist to be administered over the course of multiple doses of nausea and vomiting relief therapy as dosing is less frequent a allowing greater nausea and vomiting relief than would otherwise be achievable with a given dose of olanzapine and 5-HT3 receptor antagonist.

“Acute CINV” refers to emesis, nausea and/or vomiting that occurs within the first 24 hours of administration of one or more chemotherapeutic agents to a subject.

“Delayed CINV” refers to emesis, nausea and/or vomiting that occurs more than 24 hours after administration of one or more chemotherapeutic agents to a subject.

“Breakthrough CINV” refers to emesis, vomiting and/or nausea that occur within five days of chemotherapy administration after the use of guideline directed prophylactic antiemetic agents.

“Refractory CINV” refers to emesis, vomiting and/or nausea occurring after chemotherapy in subsequent chemotherapy cycles after guideline directed prophylactic agents have failed in earlier cycles.

“Optional” or “optionally” means that the subsequently described circumstance may or may not occur, so that the description includes instances where the circumstance occurs and instances where it does not.

The term “substantially” in reference to a certain feature or entity means to a significant degree or nearly completely (i.e. to a degree of 85% or greater) in reference to the feature or entity.

The term “about,” particularly in reference to a given quantity, is meant to encompass deviations of plus or minus 5%.

Additional definitions may also be found in the sections which follow.

### II. Chemotherapy Induced Nausea and Vomiting and Methods of Treatment

Nausea and vomiting is a human reflex against the absorption of toxins which include many chemotherapeutic agents. Chemotherapy induced nausea and vomiting (CINV) is not entirely understood, however, it is thought to have many contributing pathways which serve as potential targets for therapy. A complex condition involving multiple mechanisms, CINV has been classified into five categories. Acute CINV occurs in the first 24 hours after chemotherapy and is primarily due to the activation of the serotonin receptors in the gastrointestinal tract. Delayed CINV is nausea and/or vomiting that develops more than 24 hours after chemotherapy administration. Breakthrough CINV is vomiting and/or nausea that occurs within five days of chemotherapy administration after the use of guideline directed prophylactic antiemetic agents. Refractory CINV occurs after chemotherapy in subsequent chemotherapy cycles after guideline directed prophylactic agents have failed in earlier cycles. Anticipatory CINV may develop in patients if they experience CINV after chemotherapy administration despite prophylactic antiemetics.

The incidence and onset of CINV varies among patients, however, studies have led to the classification of chemotherapeutic agents based on their likelihood of inducing CINV. Chemotherapeutic agents can be classified as highly emetogenic (HEC) or moderately emetogenic (MEC). Highly emetic agents have a greater than 90% risk of emesis while moderately emetic agents have a 30% to 90% risk of emesis. Low risk and minimal risk emetogenic chemotherapeutics have a 10% to 30% chance and a less than 10% chance of causing CINV, respectively. Some chemotherapy regimens require no prophylaxis, whereas others require prophylaxis with medications from multiple classes. Unfortunately, some subjects still experience CINV despite administration of appropriate prophylaxis.

Extensive studies of the pathophysiology of CINV have led to the development of three classes of antiemetics commonly used for treatment and prophylactic treatment of CINV: 5-HT3 receptor antagonists, glucocorticoids, and NK-1 receptor antagonists.

Some chemotherapeutic agents cause the release of large amounts of serotonin by enterochromaffin cells in the small intestine stimulating vagal afferent nerves via the 5-HT3 receptors, thereby initiating the vomiting reflex. The development of specific 5-HT3 receptor antagonists has improved the treatment of the nausea and vomiting that often accompanies chemotherapeutic regimens. Therapeutically effective 5-HT3 receptor antagonists include, for example, granisetron, ondansetron, dolasetron, tropisetron, and palonosetron. 5-HT3 receptor antagonists have also been proven to be effective in treating post-operative nausea and vomiting (PONV).

Another mediator of vomiting pathways is substance P, a tachykinin family neuropeptide which binds to and activates the neurokinin-1 (NK-1) receptor. Accordingly, NK-1 receptor antagonists have been developed which are therapeutically effective in treating CINV and include aprepitant, fosaprepitant, netupitant, and rolapitant. NK-1 receptor antagonists are often administered in combination with 5-HT3 receptor antagonists to treat CINV.

More recently, the antipsychotic agent olanzapine has also been shown to have therapeutic value in the treatment of CINV (Brafford, 2014, J Adv Pract Oncol, 5:24-29). Olanzapine targets and blocks many different receptors such as dopaminergic, serotonergic, adrenergic, histaminergic, and muscarinic receptors. Thus, olanzapine has the advantage of targeting multiple key receptors with a single medication and has become an increasingly attractive agent for development of its use in treating CINV.

Current treatment and prophylactic treatment of CINV often involves combinations of the various antiemetics discussed above. Specifically, 5-HT3 receptor antagonists are often used in conjunction with glucocorticoid steroids and NK1 receptor antagonists. For example, acute CINV is often treated with a combination of a 5-HT3 receptor antagonist, dexamethasone and an NK-1 receptor antagonist. NK-1 receptor antagonists are also often used for treating delayed CINV. Despite the therapeutic efficacy of these regimes, there still exists a need for optimizing antiemetic therapies involving the use of olanzapine to address issues such as breakthrough CINV, reduction of side effects of currently recommended regimes, and convenience for patients undergoing chemotherapy. Of particular interest is a composition for the extended release of olanzapine and a 5-HT3 receptor antagonist from a polymeric formulation which provides therapeutic efficacy over a period of at least 5 days. In a particular embodiment, the extended release composition comprising a polyorthoester, olanzapine and granisetron. The compositions disclosed herein are useful for the treatment and/or prophylactic treatment of CINV in subjects undergoing chemotherapy, including acute, delayed, breakthrough and refractory CINV. However, it is understood that the compositions may also be useful in treating or prophylactically treating PONV in subjects in need thereof.

Thus, in one embodiment, the Applicants have discovered that the addition of olanzapine to compositions comprising a 5-HT3 receptor antagonist and a delivery vehicle is effective to provide delivery of an antiemetic combination over a period of at least about 3-5 days, and may thereby prove effective in providing extended relief from CINV. Accordingly, the systems and compositions described herein generally comprise olanzapine, a 5-HT3 receptor antagonist, and a delivery vehicle. The long-acting compositions and systems find use, for example, as drug delivery systems for treatment of emesis, nausea and/or vomiting resulting from, e.g., chemotherapy or general anesthesia used during surgical procedures. The composition components are described below, e.g., in Examples 1 and 2.

### 1. The Active Agents Olanzapine and a 5-HT3 Receptor Antagonist

In one aspect, compositions comprising the active agent olanzapine and a delivery vehicle are described. In an alternative aspect, compositions comprising olanzapine, a 5-HT3 receptor antagonist and a delivery vehicle are provided. In this section, each of the composition components is described.

### Olanzapine

Olanzapine (2-methyl-10-(4-methyl-1-piperazinyl)-4H-thieno[2,3-b][1,5]benzodiazepine) is an atypical antipsychotic agent of the thiobenzodiazepine class approved for treatment of psychotic conditions such as schizophrenia, schizophreniform diseases, and acute mania. Olanzapine is a yellow crystalline solid which is practically insoluble in water. The compound is disclosed and claimed in U.S. Pat. No. 5,229,382 to Chakrabarti et al., which is incorporated herein by reference. Olanzapine can be used both in its free base and acid addition salt forms. Acid addition salts include the pharmaceutically acceptable, non-toxic addition salts with suitable acids, such as those of inorganic acids, for example hydrochloric, hydrobromic, nitric, sulfuric or phosphoric acids, or of organic acids, such as organic carboxylic acids, for example glycolic, maleic, hydroxymaleic, fumaric, malic, tartaric, citric or lactic acid, or organic sulfonic acids for example methane sulfonic, ethane sulfonic, 2-hydroxyethane sulfonic, toluene-p-sulfonic or naphthalene-2-sulfonic acid. In addition to pharmaceutically acceptable acid addition salts, other acid addition salts include, for example, those with picric or oxalic acid, since they have potential to serve as intermediates in purification or in the preparation of other, for example, pharmaceutically acceptable, acid addition salts, or are useful for identification, characterization or purification of the free base. In addition, olanzapine can exist in pamoate salt or solvate form as taught in U.S. Pat. No. 6,617,321; or in a specific crystalline forms, such as forms I, II and III, as taught by U.S. Pat. No. 5,736,541 and U.S. Patent App. Pub. No. 20070004706; or in a solvate form as taught in U.S. Pat. No. 5,703,232; or in a hydrate form as taught by U.S. Pat. No. 6,215,895; or as co-crystalline forms as taught in U.S. Patent App. Pub. No. 20070059356, or olanzapine analogs (which includes ester analogs) as taught in U.S. Patent App. Pub. No. 20070043021. Accordingly, unless specifically noted otherwise in this application, the use of the term “olanzapine” is intended to cover the above contemplated varieties of the olanzapine.

Olanzapine has the ability to block many different receptors to provide antiemetic properties. Olanzapine targets dopaminergic (D1, D2, D3 and D4) serotonergic (5-HT2A, 5-HT2C, 5-HT3, 5-HT6), adrenergic (al), histaminergic (H1), and muscarinic (m1, m2, m3, m4) receptors. (Navari, 2015, Biomed Res Int, 2015:595894; Brafford, 2014, J Adv Pract Oncol, 5:24-29; Marek et al., 2003, Neuropsychopharmacology, 28:402-412). The present compositions comprises olanzapine or pharmaceutically acceptable salts thereof and further includes variant forms of olanzapine including but not limited to those described in U.S. Pat. Nos. 5,703,232 and 7,022,698.

The standard dosage for olanzapine for prophylaxis and treatment is 5 to 10 mg per day, with a maximum dose of 20 mg per day. Recommended starting doses are lower for women and elderly patients than for others. Olanzapine can also be given as a rescue dose: 5 mg every 4 hours as needed (Brafford, 2014, J Adv Pract Oncol, 5:24-29).

### 5-HT3 Receptor Antagonists

5-HT3 receptor antagonists are effective antiemetic agents recommended for treatment of CINV. Examples of clinically useful 5-HT3 receptor antagonists include ondansetron (Zofran®), granisetron (Kytril®), dolasetron (Anzemet®), and palonosetron (Aloxi®). Because serotonin receptor antagonists are less effective in treating anticipatory, delayed, and breakthrough CINV, the drugs can also be used in combination with other antiemetic agents to provide more comprehensive antiemetic therapy. Other known 5-HT3 receptor antagonists with potential for therapeutic value in treating nausea and vomiting include indisetron, YM-114, talipexole, azasetron, bemesetron, tropisetron, ramosetron, lerisetron, alosetron, N-3389, zacopride, cilansetron, E-3620, lintopride, KAE-393, itasetron, zatosetron, dolasetron, (±)-renzapride, (−)-YM-060, DAU-6236, BIMU-8, GK-128, Ro-93777, mirtazapine, mosapride, fabesetron, galdansetron, lurosetron, and ricasetron. Accordingly, each of the above-described 5-HT3 receptor antagonists may be combined with olanzapine in the pharmaceutical compositions described herein.

The need for antiemetic agents and therapies that address both acute and delayed emesis associated with cancer chemotherapy is highlighted by the approvals of Aloxi® and Emend®. Palonsetron (Aloxi®) is a 5-HT3 receptor antagonist with a long half-life and is approved in the U.S. for the prevention of both acute and delayed emesis. Aprepitant (Emend®) is a NK-1 receptor antagonist that belongs to a new class of anti emetic compounds and is approved for the treatment of severe and moderate CINV. Guidelines for the prevention of CINV in patients undergoing highly emetogenic chemotherapy recommend the use of aprepitant in combination with a 5-HT3 receptor antagonist and dexamethasone. While such drugs can be effective for treating acute and delayed emesis, a significant number of patients still experience emesis, indicating the need for improved antiemetic compounds and treatments.

Olanzapine in Combination with a 5-HT3 Receptor Antagonist

Pharmaceutical compositions provided by the present disclosure comprise olanzapine and a 5-HT3 receptor antagonist formulated for extended release of the active agents over a time period of at least about 3 to 7 days. In a preferred embodiment, the composition comprises olanzapine and granisetron in an amount therapeutically effective to prevent, reduce or eliminate nausea and/or vomiting in a subject in need thereof. Furthermore, the compositions are effective for the treatment of acute, delayed, breakthrough and refractive CINV resulting from highly or moderately emetogenic cancer chemotherapy.

The composition may also comprise in addition to the olanzapine and a 5-HT3 receptor antagonist, one or more additional bioactive agents.

The olanzapine is dissolved or dispersed into the composition as provided herein. The concentration of the olanzapine in the composition may vary from about 1 wt % to 20 wt %, 1 wt % to 10 wt %, 1 wt % to 9 wt %, 1 wt % to 8 wt %, 1 wt % to 7 wt %, 1 wt % to 6 wt %, 1 wt % to 5 wt %, 1 wt % to 4 wt %, 1 wt % to 3 wt %, 1 wt % to 2 wt %, 2 wt % to 10 wt %, 2 wt % to 9 wt %, 2 wt % to 8 wt %, 2 wt % to 7 wt %, 2 wt % to 6 wt %, 2 wt % to 5 wt %, 2 wt % to 4 wt %, 2 wt % to 3 wt %, 0.5 wt % to 10 wt %, 0.5 wt % to 7 wt %, 0.5 wt % to 6 wt %, 0.5 wt % to 5 wt %, 0.5 wt % to 4 wt %, 0.5 wt % to 3 wt %, 0.5 wt % to 2 wt %, 0.5 wt % to 1 wt %, and may be about 0.5 wt %, 1 wt %, 1.1 wt %, 1.2 wt %, 1.3 wt %, 1.4 wt %, 1.5 wt %, 1.6 wt %, 1.7 wt %, 1.8 wt %, 1.9 wt %, 2 wt %, 2.1 wt %, 2.2 wt %, 2.3 wt %, 2.4 wt %, 2.5 wt %, 2.6 wt %, 2.7 wt %, 2.8 wt %, 2.9 wt %, 3 wt %, 3.1 wt %, 3.2 wt %, 3.3 wt %, 3.4 wt %, 3.5 wt %, 3.6 wt %, 3.7 wt %, 3.8 wt %, 3.9 wt %, 4 wt %, 4.1 wt %, 4.2 wt %, 4.3 wt %, 4.4 wt %, 4.5 wt %, 4.6 wt %, 4.7 wt %, 4.8 wt %, 4.9 wt %, 5 wt %, 5 wt %, 5.1 wt %, 5.2 wt %, 5.3 wt %, 5.4 wt %, 5.5 wt %, 5.6 wt %, 5.7 wt %, 5.8 wt %, 5.9 wt %, 6 wt %, 6.1 wt %, 6.2 wt %, 6.3 wt %, 6.4 wt %, 6.5 wt %, 6.6 wt %, 6.7 wt %, 6.8 wt %, 6.9 wt %, 7 wt %, 7.1 wt %, 7.2 wt %, 7.3 wt %, 7.4 wt %, 7.5 wt %, 7.6 wt %, 7.7 wt %, 7.8 wt %, 7.9 wt %, 8 wt %, 8.1 wt %, 8.2 wt %, 8.3 wt %, 8.4 wt %, 8.5 wt %, 8.6 wt %, 8.7 wt %, 8.8 wt %, 8.9 wt %, 9 wt %, 9.1 wt %, 9.2 wt %, 9.3 wt %, 9.4 wt %, 9.5 wt %, 9.6 wt %, 9.7 wt %, 9.8 wt %, 9.9 wt %, or 10 wt %

The 5-HT3 receptor antagonist is dissolved or dispersed into the composition as provided herein. The concentration of the 5-HT3 receptor antagonist in the composition may vary from about 1 wt % to 20 wt %, 1 wt % to 10 wt %, 1 wt % to 9 wt %, 1 wt % to 8 wt %, 1 wt % to 7 wt %, 1 wt % to 6 wt %, 1 wt % to 5 wt %, 1 wt % to 4 wt %, 1 wt % to 3 wt %, 1 wt % to 2 wt %, 2 wt % to 10 wt %, 2 wt % to 9 wt %, 2 wt % to 8 wt %, 2 wt % to 7 wt %, 2 wt % to 6 wt %, 2 wt % to 5 wt %, 2 wt % to 4 wt %, 2 wt % to 3 wt %, 0.5 wt % to 10 wt %, 0.5 wt % to 7 wt %, 0.5 wt % to 6 wt %, 0.5 wt % to 5 wt %, 0.5 wt % to 4 wt %, 0.5 wt % to 3 wt %, 0.5 wt % to 2 wt %, 0.5 wt % to 1 wt %, and may be about 0.5 wt %, 1 wt %, 1.1 wt %, 1.2 wt %, 1.3 wt %, 1.4 wt %, 1.5 wt %, 1.6 wt %, 1.7 wt %, 1.8 wt %, 1.9 wt %, 2 wt %, 2.1 wt %, 2.2 wt %, 2.3 wt %, 2.4 wt %, 2.5 wt %, 2.6 wt %, 2.7 wt %, 2.8 wt %, 2.9 wt %, 3 wt %, 3.1 wt %, 3.2 wt %, 3.3 wt %, 3.4 wt %, 3.5 wt %, 3.6 wt %, 3.7 wt %, 3.8 wt %, 3.9 wt %, 4 wt %, 4.1 wt %, 4.2 wt %, 4.3 wt %, 4.4 wt %, 4.5 wt %, 4.6 wt %, 4.7 wt %, 4.8 wt %, 4.9 wt %, 5 wt %, 5 wt %, 5.1 wt %, 5.2 wt %, 5.3 wt %, 5.4 wt %, 5.5 wt %, 5.6 wt %, 5.7 wt %, 5.8 wt %, 5.9 wt %, 6 wt %, 6.1 wt %, 6.2 wt %, 6.3 wt %, 6.4 wt %, 6.5 wt %, 6.6 wt %, 6.7 wt %, 6.8 wt %, 6.9 wt %, 7 wt %, 7.1 wt %, 7.2 wt %, 7.3 wt %, 7.4 wt %, 7.5 wt %, 7.6 wt %, 7.7 wt %, 7.8 wt %, 7.9 wt %, 8 wt %, 8.1 wt %, 8.2 wt %, 8.3 wt %, 8.4 wt %, 8.5 wt %, 8.6 wt %, 8.7 wt %, 8.8 wt %, 8.9 wt %, 9 wt %, 9.1 wt %, 9.2 wt %, 9.3 wt %, 9.4 wt %, 9.5 wt %, 9.6 wt %, 9.7 wt %, 9.8 wt %, 9.9 wt %, 10 wt %, 11 wt %, 11.1 wt %, 11.2 wt %, 11.3 wt %, 11.4 wt %, 11.5 wt %, 11.6 wt %, 11.7 wt %, 11.8 wt %, 11.9 wt %, 12 wt %, 12.1 wt %, 12.2 wt %, 12.3 wt %, 12.4 wt %, 12.5 wt %, 12.6 wt %, 12.7 wt %, 12.8 wt %, 12.9 wt %, 13 wt %, 13.1 wt %, 13.2 wt %, 13.3 wt %, 13.4 wt %, 13.5 wt %, 13.6 wt %, 13.7 wt %, 13.8 wt %, 13.9 wt %, 14 wt %, 14.1 wt %, 14.2 wt %, 14.3 wt %, 14.4 wt %, 14.5 wt %, 14.6 wt %, 14.7 wt %, 14.8 wt %, 14.9 wt %, or 15 wt %. In a preferred embodiment of the composition described above, the 5-HT3 receptor antagonist is granisetron.

In some embodiments, the weight percent of olanzapine and the weight percent of the 5-HT3 receptor antagonist ranges from about 1 wt % to 5 wt % and 2 wt % and 7 wt %, respectively; 1 wt % to 3 wt % and 2 wt % and 5 wt %, respectively; 1 wt % to 8 wt % and 2 wt % and 10 wt %, respectively; or 1 wt % to 10 wt % and 1 wt % to 15 wt %, respectively. In other embodiments, the weight percent of olanzapine and the weight percent of the 5-HT3 receptor antagonist in the composition is about 2 wt % and 2 wt %, respectively, 2 wt % and 3 wt %, respectively; 2 wt % and 5 wt %, respectively; 2 wt % and 5 wt %, respectively; 0.5 wt % and 5 wt %, respectively; 0.5 wt % and 4 wt %, respectively; 0.5 wt % and 3 wt %, respectively; 0.5 wt % and 1 wt %, respectively; or 0.5 wt % and 1 wt %, respectively. In a preferred embodiment of the composition described above, the 5-HT3 receptor antagonist is granisetron.

### Exemplary Delivery Vehicles

The composition additionally comprises a delivery vehicle. In one embodiment, the delivery vehicle is a sustained-release vehicle, and exemplary vehicles include polymeric formulations, liposomes, microspheres, implantable device or non-polymeric formulations. Examples of these vehicles will now be described.

### Liposomes

Liposomes are small vesicles composed of lipids arranged in spherical bilayers. Liposomes are usually classified as small unilamellar vesicles (SUV), large unilamellar vesicles (LUV), multi-lamellar vesicles (MLV) or multivesicular liposomes (MVL). SUVs and LUVs, by definition, have only one bilayer, whereas MLVs contain many concentric bilayers (see, e.g., Stryer, Biochemistry, 2d Edition, W.H. Freeman & Co., p. 213 (1981)). MVLs were first reported by Kim et al. (Biochim, Biophys. Acta, 728:339-348, 1983) and contain multiple, non-concentric aqueous chambers per particle (See, U.S. Pat. Nos. 6,132,766 and 8,182,835, incorporated herein by reference in their entirety).

Liposomes suitable for use in the composition of the present invention include those composed primarily of vesicle-forming lipids. Vesicle-forming lipids can form spontaneously into bilayer vesicles in water, as exemplified by the phospholipids. The liposomes can also include other lipids incorporated into the lipid bilayers, e.g., cholesterol, with the hydrophobic moiety in contact with the interior, hydrophobic region of the bilayer membrane, and the head group moiety oriented toward the exterior, polar surface of the bilayer membrane.

The vesicle-forming lipids can have two hydrocarbon chains, typically acyl chains, and a head group, either polar or nonpolar. There are a variety of synthetic vesicle-forming lipids and naturally-occurring vesicle-forming lipids, including the phospholipids, such as phosphalidylcholine, phosphatidylethanolamine, phosphatidic acid, phosphatidylinositol, and sphingomyelin, where the two hydrocarbon chains are typically between about 14-22 carbon atoms in length, and have varying degrees of unsaturation. The above-described lipids and phospholipids whose acyl chains have varying degrees of saturation can be obtained commercially or prepared according to published methods. Other suitable lipids include glycolipids and sterols, such as cholesterol.

In one embodiment, the vesicle-forming lipid is selected to achieve a specified degree of fluidity or rigidity, to control the stability of the liposome in serum and to control the rate of release of the entrapped agent in the liposome. Liposomes may be prepared by a variety of techniques (see, e.g., Szoka, F., Jr., et al., Ann. Rev. Biophys. Bioeng. 9:467 (1980); U.S. Pat. No. 5,631,018). It will be appreciated that lipid-based delivery vehicles other than liposomes are contemplated, such as micelles and emulsions.

In one embodiment, the amide-type local anesthetic and the enolic-acid NSAID are entrapped in an aqueous space of the liposome or in a lipid layer of the liposome.

### Microspheres/Microparticles/Microcapsules

In another embodiment, the delivery vehicle is a microspheres, microparticles or microcapsules. Microspheres in the form of spherical polymer matrices with interconnected pores in which an active agent is incorporated are described, for example, in U.S. Pat. No. 4,818,542. Microparticles comprised of one or more polymers in which the active agents are incorporated or associated can be fabricated from biodegradable or non-biodegradable polymers that are suitable for in vivo use, such as poly(vinylpyrrolidone) and poly(acrylamide). The microspheres or microparticles can be administered as part of a formulation that forms a depot in situ or as part of an implant. The active agents are released from the microspheres or microparticles in a controlled fashion, to provide the desired therapeutic efficacy. In one embodiment, the sustained-release delivery vehicle is a microsphere comprised of a bioerodible or biodegradable polymer. In another embodiment, the amide-type local anesthetic and the enolic-acid NSAID are entrapped in the microsphere.

### Implantable Devices

Implantable devices with a reservoir in which the active agents are contained and controllably-released are known in the medical arts. In one embodiment, an osmotic, mechanical, or electromechanical device is provided for implantation and sustained release of the active agents. Examples of implantable devices are set forth in U.S. Pat. Nos. 7,655,254; 8,603,051; and 8,603,076 and US Publication No. 2003/0032947.

### Non-Polymeric Formulations

The delivery vehicle can also take the form of a non-polymeric, pharmaceutically acceptable carrier. For example, the non-polymeric formulation can comprise sucrose acetate isobutyrate as a non-polymeric, pharmaceutically acceptable carrier and an optional solvent, such as benzyl alcohol. The non-polymeric formulation can be a liquid. This liquid, non-polymeric formulation provides sustained local anesthesia to a subject after administration for a period of about 24-36 hours, 36-48 hours, 48-60 hours, 60-72 hours, 3-4 days or 3-5 days. In one embodiment, the delivery vehicle is comprised of between about 50-80 wt % sucrose acetate isobutyrate and between about 5-25 wt % benzyl alcohol, alternatively between 55-75 wt % sucrose acetate isobutyrate and between about 15-25 wt % benzyl alcohol, with the remainder to 100 wt % being the active agents. Exemplary non-polymeric formulations of this type are described in EP 1809329, which is incorporated herein by reference in its entirety.

In some embodiments, the liquid non-polymeric carrier is a liquid carrier material having a viscosity of about less than 50,000 mPa-s at 37° C., measured using a viscometer. Alternatively, the carrier has a viscosity of less than about 10,000 mPa-s when measured at 37° C. using a viscometer. In another embodiment, the liquid non-polymeric carrier is a liquid carrier material having a viscosity of about less than 5,000 mPa-s at 37° C. In yet another embodiment, the liquid non-polymeric carrier is a liquid carrier material having a viscosity of about less than 2,500 mPa-s at 37° C.

In another embodiment, the non-polymeric formulation is an aqueous solution.

### Polymeric Formulations

Exemplary polymeric formulations as the sustained-release delivery vehicle include those comprised of bioerodible or biodegradable polymers. The vehicle when comprised of a bioerodible or biodegradable polymer can be a solid or a semi-solid vehicle. Bioerodible and/or biodegradable polymers are known in the art, and include but are not limited to polylactides, polyglycolides, poly(lactic-co-glycolic acid) copolymers, polycaprolactones, poly-3-hydroxybutyrate, and polyorthoesters. Semisolid polymers exist either in a glassy or viscous liquid state. Semisolid polymers typically display a glass transition temperature (Tg) below room temperature. Below the Tg, semisolid polymers can be considered to exist in a glassy state, while above the Tg, the polyorthoester can be considered to exist in a liquid state. Semisolid polyorthoester polymers are not thermoplastic polymers.

In one embodiment, a bioerodible or biodegradable polymer is selected to provide a certain rate of degradation or erosion to achieve a desired release rate of the olanzapine and the 5-HT3 receptor antagonist (preferably granisetron). The delivery vehicle and active agents can be formulated to provide a semi-solid or solid composition. By way of example, in one embodiment, a semi-solid delivery vehicle comprised of a polyorthoester is provided, and some examples are set forth herein. In another embodiment, the polymeric delivery vehicle forms an implant or depot in situ.

In another embodiment, a solid delivery vehicle comprised of a biodegradable or bioerodible polymer is provided, where the solid vehicle is in the form of a rod or disk. Rods and disks are suitable for implantation into a patient, and the biodegradable or bioerodible polymer in which the active agents are incorporated can formulated to tailor the release of active agent. For example, the rod or disk can be formulated from different polymers with different rates of biodegradability or polymers of differing molecular weights can be used, as well as additives or excipients can be added to active agent-polymer matrix to tailor the rate of agent release. The rod or disk can also comprise materials commonly used in sutures and/or capable of being used in sutures, including the biodegradable polymers noted above as well as polyglactin and copolymers of glycolide with trimethylene carbonate (TMC) (polyglyconate).

In one embodiment, the delivery vehicle is comprised of a polyorthoester.

Polyorthoesters useful for the compositions provided herein are generally composed of alternating residues resulting from reaction of a diketene acetal and a diol, where each adjacent pair of diketene acetal derived residues is separated by the residue of a reacted diol. The polyorthoester may comprise α-hydroxy acid-containing subunits, i.e., subunits derived from an α-hydroxy acid or a cyclic diester thereof, such as subunits comprising glycolide, lactide, or combinations thereof, i.e., poly(lactide-co-glycolide), including all ratios of lactide to glycolide, e.g., 75:25, 65:35, 50:50, etc. Such subunits are also referred to as latent acid subunits; these latent acid subunits also fall within the more general “diol” classification as used herein, due to their terminal hydroxyl groups. Polyorthoesters can be prepared as described, for example, in U.S. Pat. Nos. 4,549,010 and 5,968,543. Exemplary polyorthoesters suitable for use in the compositions provided herein are described in U.S. Pat. No. 8,252,304.

The mole percentage of α-hydroxy acid containing subunits, R¹, generally ranges from 0 to 20 mol % of the total diol components (R¹ and R³ as provided below). In one or more embodiments, the mole percentage of α-hydroxy acid containing subunits in the polyorthoester formulation is at least about 0.01 mole percent. Exemplary percentages of α-hydroxy acid containing subunits in the polymer are from about 0 to about 50 mole percent, or from about 0 to about 25 mole percent, or from about 0.05 to about 30 mole percent, or from about 0.1 to about 25 mole percent. For example, in one embodiment, the percentage of α-hydroxy acid containing subunits in the polymer is from about 0 to about 50 mole percent. In another embodiment, the percentage of α-hydroxy acid containing subunits in the polymer is from about 0 to about 25 mole percent. In yet another particular embodiment, the percentage of α-hydroxy acid containing subunits in the polymer is from about 0.05 to about 30 mole percent. In yet another embodiment, the percentage of α-hydroxy acid containing subunits in the polymer is from about 0.1 to about 25 mole percent. As an illustration, the percentage of α-hydroxy acid containing subunits may be 0, 1, 2, 3, 4, 5, 6, 7, 8, 9, 10, 11, 12, 13, 14, 15, 16, 17, 18, 19, 20, 21, 22, 23, 24, 24, 26, 27, 28, 29 or 30 mole percent, including any and all ranges lying therein, formed by combination of any one lower mole percentage number with any higher mole percentage number.

More particularly, a poly(orthoester) for use in the compositions and delivery systems provided herein is described by the following formula:

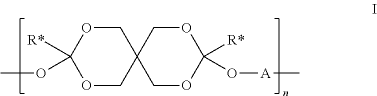

where: R* is a C₁₋₄ alkyl (e.g., C1, C2, C3 or C4 alkyl), n is an integer ranging from 5 to 400, and A in each subunit is R¹ or R³. That is, in any monomer unit

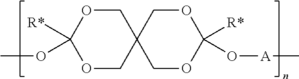

of the polymer of Formula I, A may be either R¹ or R³.

In a preferred embodiment, R* is ethyl (i.e., C2 alkyl). A subunit in accordance with Formula I, wherein R* is ethyl, corresponds to a subunit resulting from reaction of a diol as provided herein with 3,9-di(ethylidene)-2,4,8,10-tetraoxaspiro[5.5]undecane (DETOSU), a diketene acetal having the structure:

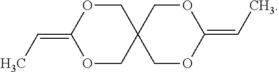

In reference to Formula I, as described previously, A may correspond to R¹. R¹ is

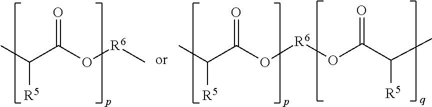

where p and q are each independently integers that range from between about 1 to 20 (e.g., are each independently selected from 1, 2, 3, 4, 5, 6, 7, 8, 9, 10, 11, 12, 13, 14, 15, 16, 17, 18, 19, and 20), each R⁵ is independently hydrogen or C₁₋₄ alkyl (e.g., is H, or C1, C2, C3, or C4 alkyl); and R⁶ is:

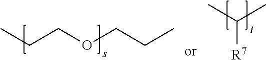

where s is an integer from 0 to 10 (e.g., is selected from, 0, 1, 2, 3, 4, 5, 6, 7, 8, 9, 10); t is an integer from 2 to 30; and R⁷ is hydrogen or C₁₋₄ alkyl (e.g., is H or C1, C2, C3, or C4 alkyl). In one or more particular embodiments, R⁷ is H. The R¹ subunits are α-hydroxy acid-containing subunits, i.e., subunits derived from an α-hydroxy acid or a cyclic diester thereof.

In reference to Formula I, A may also correspond to R³, where R³ is:

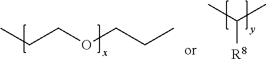

and x is an integer ranging from 1 to 100, and is, in certain particular instances, selected from 1, 2, 3, 4, and 5; y is an integer in a range from 2 to 30; and R⁸ is hydrogen or C₁₋₄ alkyl (C1, C2, C3 or C4 alkyl).

In a particular embodiment, R⁸ is H.

In some embodiments, the polyorthoester is one in which A is R¹ or R³, where R¹ is

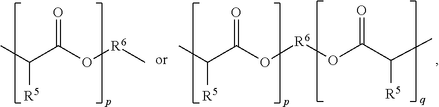

where p and q are each independently integers that range from between about 1 and 20, where the average number of p or the average number of the sum of p and q (i.e., p+q) is between about 1 and 7 (e.g., 1, 2, 3, 4, 5, 6, 7) in at least a portion of the monomeric units of the polymer, x and s are each independently an integer ranging from 0 to 10; and t and y are each independently an integer ranging from 2 to 30. In one or more particular embodiments, R⁵ is H.

Additional particular polyorthoesters are those in which A is R¹ or R³, where R¹ is

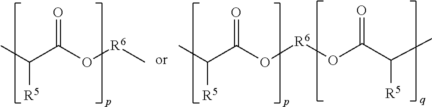

and p and q are each independently integers that vary from between about 1 and 20, or between about 1 and 15, or between about 1 and 10, where the average number of p or the average number of the sum of p and q (i.e., p+q) is between about 1 and 7 in at least a portion of the monomeric units of the polymer. Additionally, particular ranges of x and s (in reference to the preferred embodiment above or in reference to any polyorthoester as provided herein) are those in which each is independently an integer ranging from 0 to 7 or from 1 to 5. Similarly, particular ranges for t and y are those in which each independently varies from 2 to 10.

Preferred polyorthoesters are those in which R⁵ is hydrogen or methyl.

In certain preferred embodiments, s and x are each independently selected from 1, 2, 3, 4, 5, 6, 7 and 8. In some preferred embodiments, s is 2. In some other preferred embodiments, x is 2.

An exemplary polyorthoester comprises alternating residues of 3,9-diethyl-3,9-2,4,8,10-tetraoxaspiro[5.5]undecane-3,9-diyl and A:

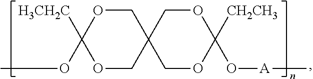

where A is as described above.

Polyorthoesters such as those described herein can be prepared by reacting an illustrative diketene acetal, 3,9-di(ethylidene)-2,4,8,10-tetraoxaspiro[5.5]undecane (DETOSU),

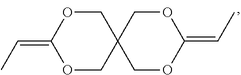

with one or more diols as described above, such as HO—R¹—OH or HO—R³—OH. Illustrative diols include oligoethylene glycols such as triethylene glycol (TEG), oligoethylene glycols modified at one or both termini with an α-hydroxy acid such as an oligoethylene glycol diglycolide or an oligoethylene glycol dilactide, organic diols having a hydrocarbyl core of from 2 to 30 carbon atoms such as 1,6-hexanediol, 1,10-decanediol, cis/trans 1,4-cyclohexane dimethanol, para-menthane-3,8-diol, 1,4-butanediol, 1,5-pentanediol, 1,7-heptanediol, 1,8-octanediol, 1,10-decanediol, 1,12-dodecanediol, and cyclic equivalents thereof, where the hydroxyl groups can be at any two positions within the cycloalkyl or alkylene ring. An organic diol can possess from 2 to 20 carbon atoms. The organic diol can be linear, branched or cyclic, and may also be saturated or unsaturated. Generally, unsaturated diols will possess from 1-3 elements of unsaturation. A preferred poly(orthoester) will contain from about from 10 to 50 total mole percent of subunits derived from one or more organic diols having a hydrocarbyl core.

Diols such as HO—R¹—OH are prepared as described in U.S. Pat. No. 5,968,543 and in Heller et al., J. Polymer Sci., Polymer Letters Ed. 18:293-297 (1980). For example, a diol of the formula HO—R¹—OH comprising a polyester moiety can be prepared by reacting a diol of the formula HO—R³—OH with between 0.5 and 10 molar equivalents of a cyclic diester of an α-hydroxy acid such as lactide or glycolide, and allowing the reaction to proceed at 100° C.-200° C. for about 12 hours to about 48 hours. Suitable solvents for the reaction include organic solvents such as dimethylacetamide, dimethyl sulfoxide, dimethylformamide, acetonitrile, pyrrolidone, tetrahydrofuran, and methylbutyl ether. Although the diol product is generally referred to herein as a discrete and simplified entity, e.g., TEG diglycolide (and diol reaction products such as TEG diglycolide), it will be understood by those of skill in the art that due to the reactive nature of the reactants, e.g., ring opening of the glycolide, the diol is actually a complex mixture resulting from the reaction, such that the term, TEG diglycolide (or any other term referring a similar product), generally refers to the average or overall nature of the product.

A preferred polyorthoester is prepared by reacting 3,9-di(ethylidene)-2,4,8,10-tetraoxaspiro[5.5]undecane (DETOSU) with one or more reactive diols. Generally, the polyorthoester is prepared by reacting DETOSU with two or more reactive diols under anhydrous conditions. A preferred polyorthoester is prepared by reacting DETOSU with triethylene glycol and triethylene glycol diglycolide as described in U.S. Pat. No. 8,252,305. A particular polyorthoester prepared from DETOSU-triethylene glycol-triethylene glycol diglycolide possesses the following molar ratios of components: 90:80:20, although the relative ratios of components can be suitably varied as described above.

A polyorthoester formed by the reaction of DETOSU with TEG and TEG diglycolide can generally be described as possessing the following subunits, where R¹ corresponds to the diolate portion derived from triethylene glycol diglycolide (formed by reaction of glycolide with TEG) and R³ corresponds to the diolate portion derived from triethylene glycol:

where A is R¹, and R¹ is

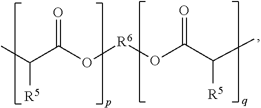

where R⁵ is H and R⁶ is

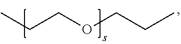

the resulting component of the polyorthoester is:

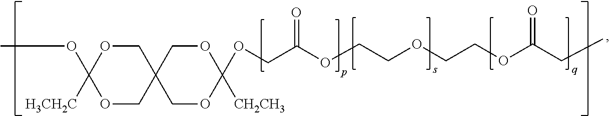

where the sum of p and q is, on average, 2 and s is 2; and when A is R³, and R³ is

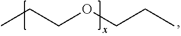

where x is 2, the resulting subunit or component of the polyorthoester is

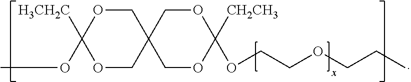

Structures corresponding to polyorthoesters prepared from the various α-hydroxy acid-containing subunits and additional diols described herein can be readily envisioned.

Exemplary polyorthoesters possess a weight average molecular weight of about 1000 Da to about 200,000 Da, for example from about 2,500 Da to about 100,000 Da or from about 3,500 Da to about 20,000 Da or from about 4,000 Da to about 10,000 Da or from about 5,000 Da to about 8,000 Da. Illustrative molecular weights, in Da, are 2500, 5000, 5500, 6000, 6500, 7000, 7500, 8000, 8500, 9000, 9500, 10,000, 20,000, 30,000, 40,000, 50,000, 60,000, 70,000, 80,000, 90,000, 100,000, 120,000, 150,000, 175,000 and 200,000, and ranges therein, wherein exemplary ranges include those formed by combining any one lower molecular weight as described above with any one higher molecular weight as provided above, relative to the selected lower molecular weight.

In one embodiment, the poly(orthoesters) described in this section are semi-solids both at room temperature and at temperatures above room temperature. In one embodiment, polyorthoesters containing 80 to 100 mole % R³, where R³ is

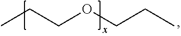

where x is 2, are semisolid polymers at both room temperature and at temperatures above room temperature. Semisolid polymers exist either in a glassy or viscous liquid state. Semisolid polymers typically display a glass transition temperature (Tg) below room temperature. Below the Tg, semisolid polymers can be considered to exist in a glassy state, while above the Tg, the polyorthoester can be considered to exist in a liquid state. Semisolid polyorthoester polymers are not thermoplastic polymers.

Generally, polyorthoesters in accordance with any one of the following formulae, Formula I, Formula II, Formula III or Formula IV, are suitable for use in the compositions and/or delivery vehicles provided herein:

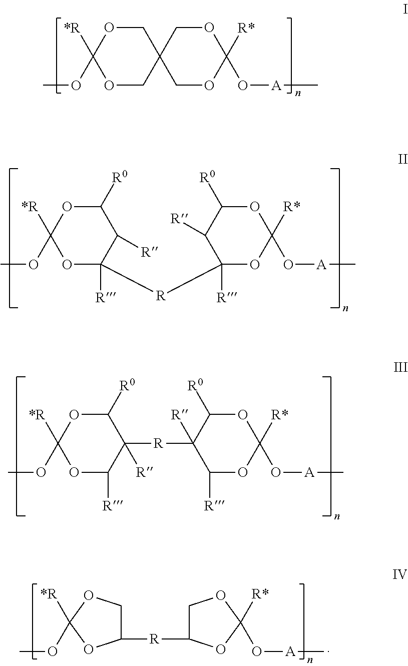

In reference to Formulas I-IV,
R is a bond, —(CH₂)ₐ—, or —(CH₂)_{b}—O—(CH₂)_{c}—; where a is an integer from 1 to 12 (e.g., selected from 1, 2, 3, 4, 5, 6, 7, 8, 9, 10, 11, and 12), and b and c are independently integers from 1 to 5 (e.g., selected from 1, 2, 3, 4, and 5);
R* is a C₁₋₄ alkyl;
R^{o}, R″ and R′″ are each independently H or C₁₋₄ alkyl;
n is an integer of at least 5; and
A is a diol.

For example, the compositions and delivery systems described herein may be comprised of a polyorthoester of Formula I, Formula II, Formula III or Formula IV, where:

R is a bond, —(CH₂)ₐ—, or —(CH₂)_{b}—O—(CH₂)_{c}—; where a is an integer of 1 to 12, and b and c are independently integers of 1 to 5;
R* is a C₁₋₄ alkyl;
R^{o}, R″ and R′″ are each independently H or C₁₋₄ alkyl;
n is an integer of at least 5; and
A is R¹, R², R³, or R⁴, where
R¹ is an α-hydroxy acid containing subunit as described in the preceding paragraphs;
R⁵ is hydrogen or C₁₋₄ alkyl (e.g., methyl, ethyl, propyl, butyl, isopropyl, isobutyl, sec-butyl); and
R⁶ is selected from the group consisting of:

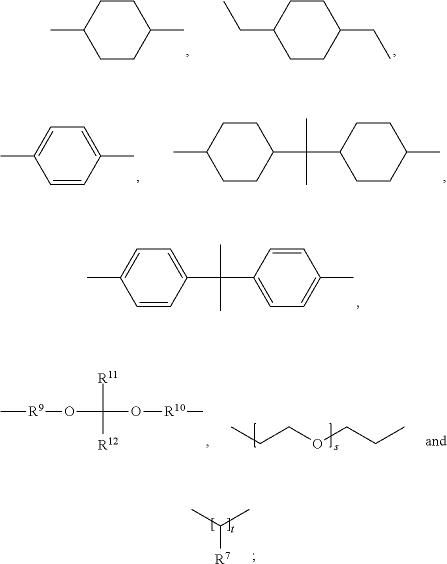

where:
s is an integer ranging from 0 to 10;
t is an integer ranging from 2 to 30; and
R⁷ is hydrogen or C₁₋₄ alkyl;

### R² is:

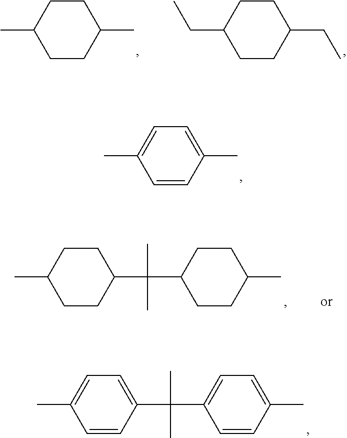

### R³ is:

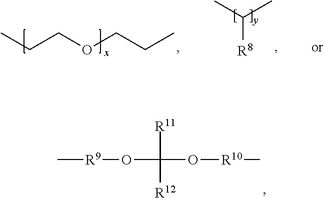

where:
x is an integer ranging from 0 to 200;
y is an integer ranging from 2 to 30;
R⁸ is hydrogen or C₁₋₄ alkyl;
R⁹ and R¹⁰ are independently C₁₋₁₂ alkylene;
R¹¹ is hydrogen or C₁₋₆ alkyl and R¹² is C₁₋₆ alkyl; or R¹¹ and R¹² together are C₃₋₁₀ alkylene; and
R⁴ is the residue of a diol containing at least one functional group independently selected from an amide, an imide, a urea, and a urethane (carbmate) group.
In certain instances, the polyorthoester is one according to any one of Formulae I-IV in which A is R¹, R³, or R⁴, where
R³ is selected from:

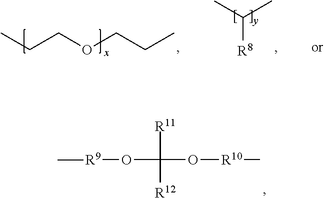

where:
x is an integer of 0 to 100;
y is an integer of 2 to 30;
R⁸ is hydrogen or C₁₋₄ alkyl;
R⁹ and R¹⁰ are independently C₁₋₁₂ alkylene;
R¹¹ is hydrogen or C₁₋₆ alkyl and R¹² is C₁₋₆ alkyl; or R¹¹ and R¹² together are C₃₋₁₀ alkylene;
R⁴ is a residual of a diol containing at least one functional group independently selected from amide, imide, urea and urethane groups; and R⁵ is hydrogen or C₁₋₄ alkyl.

In one particular embodiment of the polyorthoester, the fraction of the A units that are of the formula R¹ is between 0 and 20 mole percent.

One exemplary polyorthoester is described by formula I, II, III or IV, where:

none of the units have A equal to R²;

### R³ is:

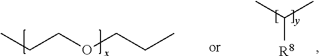

where:
x is an integer of 1 to 100;
y is an integer of 2 to 30; and

### R⁶ is:

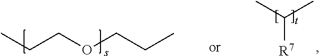

where:
s is an integer of 1 to 10;
t is an integer of 2 to 30; and
R⁵, R⁷, and R⁸ are independently hydrogen or methyl.

An additional representative polyorthoester of Formula I, II, III or IV, is one in which R³ and R⁶ are both —(CH₂—CH₂—O)₂—(CH₂—CH₂)—; R⁵ is methyl; and where p and q are independently 0, 1 or 2.

In another embodiment of a polyorthoester of Formula I, II, III or IV, R³ and R⁶ are both —(CH₂—CH₂—O)₉—(CH₂—CH₂)—; R⁵ is methyl; and p or the sum of p and q is on average 2.

In another variation, the polyorthoester is of Formula I, II, III or IV, R is —(CH₂)_{b}—O—(CH₂)_{c}—; where b and c are both 2; R* is a C₂ alkyl.

Additional representative polyorthoesters of Formula I, II, III or IV, are those in which R⁵ is hydrogen or methyl; R⁶ is

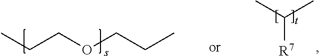

where s is an integer from 1 to 10, e.g., preferably selected from 1, 2, 3, or 4; t is an integer from 2 to 30, particularly selected from 2, 3, 4, 5, 6, 7, 8, 9 and 10; R⁷ is hydrogen or methyl; and R³ is

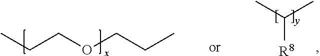

where x is an integer from 1 to 10, e.g., preferably selected from 1, 2, 3, or 4; y is an integer from 2 to 30, particularly selected from 2, 3, 4, 5, 6, 7, 8, 9 and 10; R⁸ is hydrogen or methyl; R⁴ is selected from a residue of an aliphatic diol having from 2-20 carbon atoms (e.g., selected from 2, 3, 4, 5, 6, 7, 8, 9, 10, 11, 12, 13, 14, 15, 16, 17, 18, 19, and 20 carbon atoms), preferably having from 2 to 10 carbon atoms, interrupted by one or two amide, imide, urea, or urethane groups. Preferably, the proportion of subunits in the polyorthoester in which A is R¹ is from about 0.01-50 mole percent. In certain instances, the proportion of subunits in the polyorthoester in which A is R¹ is from about 0 to about 30 mole percent, or from about 0.1 to 25 mole percent. Illustrative mole percentages include 10, 15, 20 and 25 mole percent of subunits in the polyorthoester in which A is R¹. In one embodiment, the mole percent is 20. Additionally, in one or more embodiments, the proportion of subunits in which A is R² is less than about 20 percent, preferably less than about 10 percent, or more preferably less than about 5 percent, and the proportion of subunits in which A is R⁴ is less than 20 percent, preferably less than about 10 percent or more preferably less than 5 percent.

The polyorthoester, as shown in Formula I, Formula II, Formula III and Formula IV, in certain embodiments, is one of alternating residues of a diketene acetal and a diol, with each adjacent pair of diketene acetal residues being separated by the residue of one polyol, such as a diol.

Methods of manufacturing the polyorthoesters are well known in the art, and are described, e.g., in U.S. Pat. Nos. 6,613,355 and 8,252,304.

### Optional Solvents and Excipients

The composition may additionally comprise one or more pharmaceutically acceptable excipients, and some examples are now set forth.

In the embodiment wherein the delivery vehicle is a polymeric formulation, and in particular where the polymer is a polyorthoester, the delivery vehicle may optionally comprise an organic acid, such as described in U.S. application Ser. No. 14/691,491, filed Apr. 20, 2015, to facilitate the release of the active agent from the vehicle or composition, in particular, during the early stages of delivery (e.g., days 1-3 post-administration). Generally, the organic acid is a carboxylic acid. Most suitable are organic acids having a molecular weight less than about 300 daltons. Representative organic acids include, e.g., fumaric or maleic acid, ethanoic acid, propanoic acid, butanoic acid, pentanoic acid, hexanoic acid, heptanoic acid, benzoic acid, salicylic acid and acetyl salicylic acid, oxalic acid, malonic acid, succinic acid, glutaric acid, adipic acid, pimelic acid, and so forth.

The vehicle may comprise from about 0-80 mole percent of a mono-carboxylic acid, or from about 0-40 mole percent of a di-carboxylic acid, or from about 0 to 25 a tri-carboxylic acid based upon the concentration of basic active agent, for example, bupivacaine base. The amount of the organic acid additive comprised in the vehicle will depend, at least in part, upon the identity of the particular active agent, the amount of active agent contained in the vehicle, the particular polyorthoester, amount thereof, and desired delivery profile.

As discovered by the Applicants, for a given organic acid, vehicles comprising a greater amount of the organic acid exhibit a faster release rate which is typically most pronounced during the first 1-3 days following administration.

In another embodiment, the delivery vehicle in the form of a semi-solid polyorthoester polymeric formulation may also contain one or more liquid excipients. Preferably, the excipient is a pharmaceutically-acceptable polyorthoester compatible liquid excipient. Such excipients are liquid at room temperature and are readily miscible with polyorthoesters. Exemplary polyorthoester compatible liquid excipients include both protic and aprotic solvents. Protic liquid excipients include polyethylene glycol having a molecular weight between about 200 Da and 4,000 Da, or a polyethylene glycol derivative or co-polymer having a molecular weight between about 200 Da and 4,000 Da, e.g., an end-capped PEG such as monomethoxypolyethylene glycol, or a mono-, di- or triglyceride of a C2-19 aliphatic carboxylic acid or a mixture of such acids, and alkoxylated tetrahydrofurfuryl alcohols. Additional suitable liquid excipients include C1-C4 alkyl ethers of alkoxylated tetrahydrofurfuryl alcohols, and C2-19 aliphatic carboxylic acid esters, or the like. A one embodiment, an excipient for semi-solid vehicles is monomethoxy-PEG, having a molecular weight selected from 400, 450, 500, 550, 600 and 650.

Additional liquid excipients include aprotic solvents. Aprotic solvents suitable for use, as well as exemplary polyorthoester vehicles comprising an aprotic solvent are described in U.S. Patent Application Publication No. 2014/0275046, which is incorporated herein by reference in its entirety. Examples of hydrophilic biocompatible, aprotic organic solvents include, for example, amides such as N-methyl-2-pyrrolidone (NMP), 2-pyrrolidone, N-ethyl-2-pyrrolidone, N-cycylohexyl-2-pyrrolidone, dimethyl acetamide, and dimethyl formamide; esters of monobasic acids such as methyl lactate, ethyl lactate, and methyl acetate; sulfoxides such as dimethyl sulfoxide and decylmethylsulfoxide; lactones such as e-caprolactone and butyrolactone; ketones such as acetone and methyl ethyl ketone; and ethers such as dimethyl isosorbide and tetrahydrofuran. A preferred liquid excipient is the aprotic solvent dimethyl sulfoxide.

The delivery vehicle in the form of a semi-solid polymeric formulation can be prepared by mixing or blending the active agents, the polymer, such as the polyorthoester, an optional polymeric/polyorthoester-compatible liquid, and any other additional additives or excipients as desired. The mixing or blending can be performed by any suitable method, generally at a temperature less than about 50° C., e.g., at room temperature, although in certain instances, depending upon the nature of the materials, mixing or blending may be carried out at higher temperatures, e.g., from about 25 to 100° C. The mixing or blending is generally carried out in the absence of additional solvents, to obtain a homogeneous, flowable and non-tacky vehicle at room temperature.

The polymeric-compatible liquid is typically added to the compositions in an amount ranging from about 10 percent to about 30 percent by weight, relative to the total weight of the of the delivery vehicle. The solvent may be present in the composition in an amount ranging from about 8 percent to about 30 percent by weight. In other embodiments, the solvent is present in the composition in an amount ranging from about 8-25 wt %, 10-25 wt %, 15-25 wt %, 8-20 wt %, 10-20 wt %, 10-15 wt %, 15-20 wt %, 15-25 wt %, 10-30 wt %, 15-30 wt %, 20-25 wt %, or 10-30 wt %. The concentration of the solvent allows for the level of polymer (e.g., polyorthoester) in the composition to range from about 60-90 wt %, 70-90 wt %, 70-85 wt %, 75-90 wt %, 75-90 wt %, 80-90 wt % 70-85 wt % or 70-80 wt %.

In other embodiments, the solvent may be present in the composition in an amount, relative to the combined amount of polymer and solvent in the composition, ranging from about 10-25 wt %, 10-20 wt %, 15-20 wt %, 15-25 wt %, 5-15 wt %, 10-20 wt %, or 10-15 wt %. The concentration of solvent may allow for the level of polymer in the composition to range from about 70-90 wt %, 75-85 wt %, 80-90 wt %, or 75-90 wt %, or 75-90 wt % relative to weight of the polymer and solvent in the composition.

The polymer/solvent concentrations permit the liquid polymer/solvent compositions to be easily injected with standard syringes and small gauge needles (e.g., about 18-26 gauge) unlike liquid polymer formulations previously described, for example, which in some embodiments, unlike the present compositions, require the addition of a particulate material to achieve an acceptable viscosity for injection with a syringe and needle. The compositions of the invention can be administered into the body of a human subject or animal such as a dog, cat, horse, etc.

The rate of release of the active agent (e.g., drug) can be controlled by adjusting the composition and amount of the polymer and/or by the selection and quantity of the optional additives/excipients. The chemical structure of the polymer (i.e., the type of monomer used or the ratio of monomers for copolymers or terpolymers, the end groups on the polymer chains, and the molecular weight of the polymer) will determine the hydrophilicity or lipophilicity of the polymer material as well as contribute to the degradation time of the polymer depot. More hydrophilic polymers (e.g., polyorthoesters wherein the diol monomer is hydrophilic, e.g., triethylene glycol, tetraethylene glycol, or polyethylene glycol and the like) are used in applications where faster release rates and shorter durations of release are needed. The composition includes the delivery vehicle and the active agents in an amount effective to provide the desired therapeutic effect over the release period.

While the singular form is used to describe the polyorthoester and other composition components in this application, it is understood that more than one polyorthoester and/or more than one 5-HT3 receptor antagonist selected from the group described above may be used in the delivery system. In some embodiments of the herein described methods and compositions, the compositions further comprise one or more additional excipients. In one embodiment, a preferred excipient is one that does not influence the release of the active agents from the composition.

It is also understood that while not required, other pharmaceutically acceptable inert agents such as coloring agents and preservatives may also be incorporated into the composition.

The instant compositions may be injected or applied with standard syringes and needles (e.g., about 16 gauge), or may be applied with, e.g., a spray applicator. The compositions may be injected subcutaneously, intradermally or intramuscularly. The compositions may be applied using various methods known in the art, including by syringe, injectable or tube dispenser.

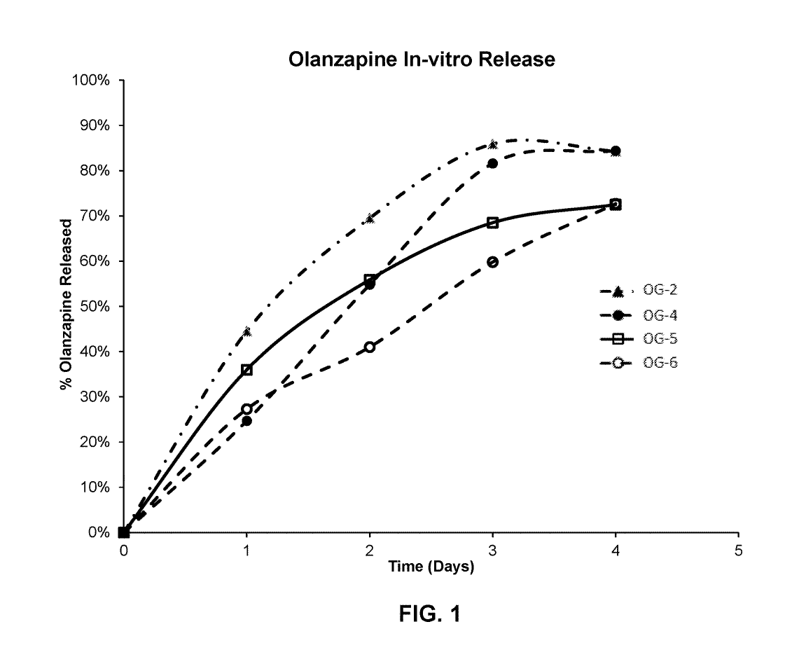

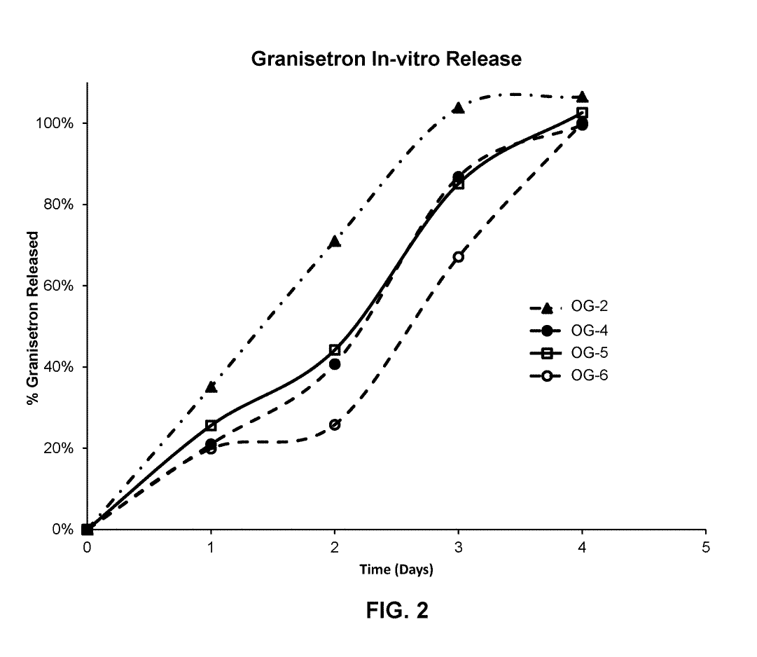

Studies were performed that illustrate modulation of release of active agents from a polyorthoester sustained release delivery vehicle. In one study (see Example 1), compositions comprising granisetron, olanzapine, a polyorthoester polymer, and a liquid excipient were prepared using the exemplary polyorthoester (POE) of Formula I described above and the DMSO as the liquid excipient. The formulations are provided in Table 1-1 of Example 1. Release of olanzapine and granisetron from the formulations was measured in an in vitro dissolution release apparatus as described in Example 2. The results are provided in Tables 2-1 and 2-2 and are shown in FIGS. 1 and 2.

FIG. 1 shows the percent of granisetron released as a function of time, in hours, in formulations comprising 2 wt % granisetron, 5 wt % olanzapine, 23 wt % DMSO and 70 wt % POE (closed triangles; OG-02); 2 wt % granisetron, 3 wt % olanzapine, 15 wt % DMSO and 80 wt % POE (closed circles; OG-4); 2 wt % granisetron, 3 wt % olanzapine, 17 wt % DMSO and 78 wt % POE (open squares; OG-05); and 2 wt % granisetron, 3 wt % olanzapine, 20 wt % DMSO and 75 wt % POE (open circles; OG-06). The percent release of granisetron varied at least at the 2 day and 3 day time points, with release of at least about 25%, 40% or 70% granisetron at 2 days after immersion of the composition in saline and release of at least about 65%, 85% or 95% at 3 days after immersion in saline.

FIG. 2 shows the percent of olanzapine released as a function of time, in hours, in formulations comprising 2 wt % granisetron, 5 wt % olanzapine, 23 wt % DMSO and 70 wt % POE (closed triangles; OG-02); 2 wt % granisetron, 3 wt % olanzapine, 15 wt % DMSO and 80 wt % POE (closed circles; OG-4); 2 wt % granisetron, 3 wt % olanzapine, 17 wt % DMSO and 78 wt % POE (open squares; OG-05); and 2 wt % granisetron, 3 wt % olanzapine, 20 wt % DMSO and 75 wt % POE (open circles; OG-06). The percent release of olanzapine varied at least at the 2 day and 3 day time points, with release of at least about 40%, 50% or 70% olanzapine at 2 days after immersion of the composition in saline and release of at least about 60%, or 80% at 3 days after immersion in saline.

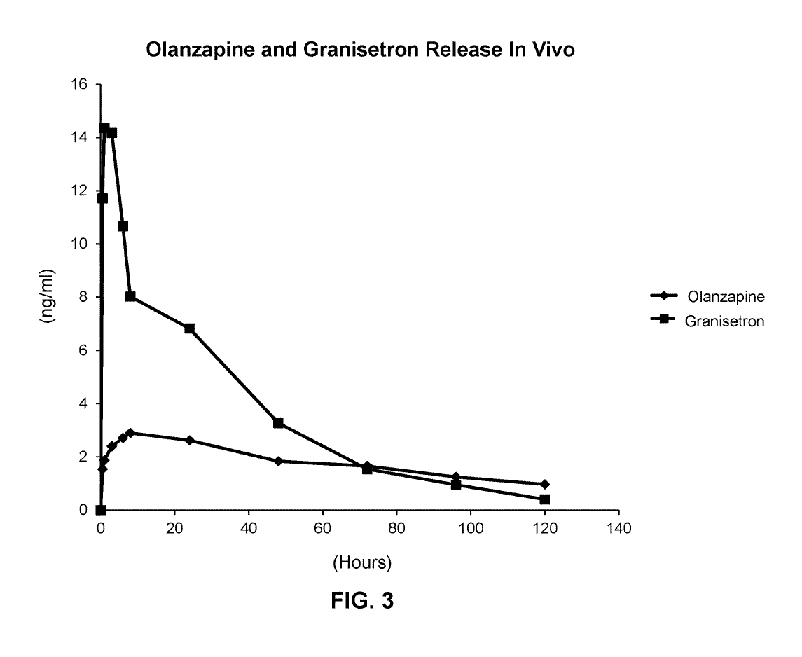

In vivo studies were performed in which dogs were administered a polymer formulation containing about 5 mg granisetron and 7.5 mg olanzapine as described in Example 3. The administered composition comprised about 2 wt % granisetron, about 3 wt % olanzapine, about 15 wt % DMSO and about 80 wt % polyorthoester of formula I. The concentration of granisetron and olanzapine in the plasma at time points varying from 0 to 120 hours (5 days) after administration was determined by LC/MS/MS. The results are provided in Tables 3-1 and 3-2 and illustrated in FIG. 3.

### Polymer Compositions Comprising Olanzapine and a 5-HT3 Receptor Antagonist

As described herein, polymer compositions comprising olanzapine and a 5-HT3 receptor antagonist provide extended releases of the active agents over a time period of at least 5 days, and can be formulated to provide release over a time period of about 1 to 7 days. Based upon the disclosures and guidance provided herein, a person having ordinary skill in the art would understand that the combination of olanzapine and a 5-HT3 receptor antagonist would also be more effective than an equal amount of the olanzapine or the 5-HT3 receptor antagonist administered alone. In a particular embodiment, the 5-HT3 receptor antagonist in the polymer composition is granisetron. In a more particular embodiment, the polymer composition comprises olanzapine and granisetron.

### 2. Methods of Treatment

The compositions provided can be used, for example, in treating, reducing or preventing nausea and/or vomiting in a patient such as a patient undergoing chemotherapy (e.g., CINV). In some embodiments, the compositions can be used to treat or prevent nausea and/or vomiting in a patient who has undergone general anesthesia during a surgical operation (e.g., PONV). Accordingly, methods of treating or prophylactically treating a patient in need thereof are provided. In some embodiments, a method for treating or prophylactically treating CINV is provided. In other embodiments, a method for treating or prophylactically treating PONV is provided. In still other embodiments, provided is a method for extending the period of relief from nausea and/or vomiting provided by administration to the patient a polyorthoester composition comprising olanzapine and a 5-HT3 receptor antagonist by incorporating therein, both active agents, to thereby provide a composition capable of providing effective relief for a period of time that is extended over that of the same composition comprising only olanzapine or only the 5-HT3 receptor antagonist. In preferred embodiments, the 5-HT3 receptor is granisetron.

The composition as described herein comprising the polyorthoester, olanzapine and 5-HT3 receptor antagonist can be administered prior to administration of highly or moderately emetogenic cancer chemotherapeutics. For example, the composition can be administered less than about 2 hours, 1 hour or 30 minutes prior to administration of one or more chemotherapeutic agents. In some embodiments, the composition is administered during or immediately following completion of the administration of the chemotherapeutic agent(s). Alternatively, the composition is administered 12 hours, or about 24 hours to 48 hours following completion of the administration of the chemotherapeutic agent(s).

In a situation of refractive or breakthrough CINV, the composition comprising the polyorthoester, olanzapine and 5-HT3 receptor antagonist is administered to a subject

Also provided is a method for extending the period of relief from nausea and/or vomiting provided by administration to the patient a polyorthoester composition comprising olanzapine and a 5-HT3 receptor antagonist by incorporating therein, both active agents, to thereby provide a composition capable of providing effective relief for a period of time that is extended over that of the same composition comprising only olanzapine or only the 5-HT3 receptor antagonist. In one embodiment, the patient is at risk of suffering from acute or delayed CINV. In another embodiment, the subject has experienced or is experiencing breakthrough or refractive CINV. In a preferred embodiment, the subject who is at risk of suffering from acute or delayed CINV or has experienced or is experiencing breakthrough or refractive CINV is administered the polyorthoester composition comprising olanzapine and granisetron.

In particular, the composition comprising a combination of olanzapine and a 5-HT3 receptor antagonist is effective to prevent, reduce, or provide relief from CINV for a period of time ranging from about 1 day to at least about 2 days or at least about 3 days or at least about 4 days or at least about 5 days following administration of a highly emetogenic or moderately emetogenic chemotherapeutic, i.e., is a long-acting composition for prevention, reduction, or relief, rather than a short-acting composition. In other embodiments, the composition prevents, reduces, or provides relief from CINV for a period of up to about 5 days, up to about 6 days, or up to about 7 days.

In some embodiments, the composition is effective to provide measurable plasma concentrations of the olanzapine and 5-HT3 receptor antagonist for a period of up to 5 days following administration. In preferred embodiments, the 5-HT3 receptor is granisetron.

In particular embodiments, the composition is effective to release a significant portion of both the olanzapine and 5-HT3 receptor antagonist from the composition, such that 80% by weight or more of both drugs are released over a period of about 5 days or up to at least about 5 days. In one embodiment, both drugs are released for a time period of between at least about 1 day to up to about 5 days, and in another embodiment for a period of between about 1 to 5 days or from about 2 to 3 days, or for at least about 3 days. Although in some cases the olanzapine may be released from the composition in approximately the same amount and over approximately the same time frame as the 5-HT3 receptor antagonist.

In another aspect, provided is a method of treatment, the method comprising dispensing from a needle a composition comprising olanzapine, a 5-HT3 receptor antagonist, and a polyorthoester, to thereby achieve a controlled release of both the olanzapine and 5-HT3 receptor antagonist from the composition, wherein 80% by weight or more of both drugs are released over a period of about 5 days.

In another embodiment, the compositions provided herein are for use in a method of prophylactic treatment or treatment of nausea and/or vomiting such as that associated with CINV or PONV to a patient in need thereof. The treatment includes administering to a patient a composition as set forth herein, e.g., comprising olanzapine and 5-HT3 receptor antagonist and a delivery vehicle, where in some embodiments, the delivery vehicle is a polyorthoester and the 5-HT3 receptor antagonist is granisetron. The method provides rates of release of both the olanzapine and the 5-HT3 receptor antagonist, as well as accompanying pharmacokinetic profiles of each effective for reducing or preventing emesis, nausea and/or vomiting over an extended period following application. Local administration can be, e.g., intramuscularly or subcutaneously.

In one embodiment, the extended period is for at least about 5 days. In another embodiment, the extended period is for up to about 5 days. In still another embodiment, the extended period from about 1 day to at least about 5 days or from about 1 day to up to about 5 days. In yet another embodiment, the extended period is for about 3 days.

In the methods, in one embodiment, about 80% by weight or more of both drugs are released from the pharmaceutical composition over a period of about 5 days. The composition, in one embodiment, is effective to prevent or provide significant relief from emesis, nausea and/or vomiting for at least about 5 days following administration of the composition.

A method is provided, where the method comprises providing a composition as described herein, and instructing that the composition be administered to the patient for prevention of acute and/or delayed nausea and vomiting associated with the initial and repeat courses of highly emetogenic or moderately emetogenic cancer chemotherapy for an extended period. Alternatively, a method is provided, where the method comprises providing a composition as described herein, and instructing that the composition be administered to the patient for prevention and treatment of postoperative nausea and vomiting. In some embodiments, the patient is an adult. In one embodiment, the extended period is for at least about 5 days. In another embodiment, the extended period is for up to about 5 days. In still another embodiment, the extended period from about 1 day to at least about 5 days or from about 1 day to up to about 5 days. In yet another embodiment, the extended period is for about 3 days.

In terms of administration for any of the methods described herein, the compositions may be injected, instilled, or applied with standard syringes and needles (e.g., about 16 gauge), or may be applied with, e.g., a spray applicator. The compositions may be injected subcutaneously, intradermally or intramuscularly. The compositions may be applied using various methods known in the art, including by syringe, injectable or tube dispenser.

### Aspects and Embodiments

Aspects and embodiments of the delivery systems, compositions, and related methods as provided herein are set forth below.

In a first aspect, provided herein is a pharmaceutical composition, comprising olanzapine and a delivery vehicle.

In a second aspect, provided herein is a pharmaceutical composition, comprising olanzapine, a 5-HT3 receptor antagonist and a delivery vehicle.

In a third aspect, provided herein is a method of treatment, comprising administering to the subject a pharmaceutical composition comprising olanzapine, a 5-HT3 receptor antagonist and a delivery vehicle.

In a 1^{st} embodiment related to the composition of the first, second or third aspects above, the composition is an aqueous based solution.

In a 2^{nd} embodiment related to the composition of the first, second or third aspects above, the delivery vehicle is a sustained release delivery vehicle.

In a 3^{rd} embodiment related to the third embodiment above, the sustained-release delivery vehicle is a polymeric composition, a liposomal composition, a microsphere composition, a non-polymeric composition or an implantable device.

In a 4^{th} embodiment related to the composition of the first, second or third aspects above, and any one of embodiments 1-3, the composition is injectable.

In a 5^{th} embodiment related to the composition of the first, second or third aspects above, and any one of embodiments 1-4, the composition is suitable for administration as an intramuscular, intravenous, transdermal, or subcutaneous injection.

In a 6^{th} embodiment related to the composition of the first, second or third aspects above, and any one of embodiments 1-5, the composition has a viscosity of less than 10,000 mPa-s when viscosity is measured at 37° C. using a cone and plate viscometer.

In a 7^{th} embodiment related to the composition of the first, second or third aspects above, and any one of embodiments 1-6, the composition has a viscosity of less than 10,000 mPa-s when viscosity is measured at 25° C. using a cone and plate viscometer.

In an 8^{th} embodiment related to the composition of the first, second or third aspects above, and any one of embodiments 2-7, the sustained release delivery vehicle is a polymeric formulation in the form of a semi-solid polymer formulation comprising a polymer, the 5-HT3 receptor antagonist and the olanzapine.

In a 9^{th} embodiment related to the composition of the first, second or third aspects above, and any one of embodiments 2-8, the delivery vehicle comprises a polyorthoester which has a structure defined by formula I, formula II, formula III or formula IV:

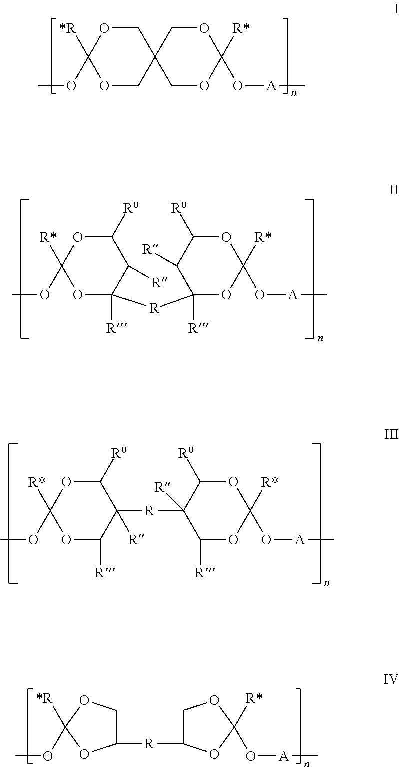

where:
R is a bond, —(CH₂)ₐ—, or —(CH₂)_{b}—O—(CH₂)_{c}—; where a is an integer from 1 to 12 (e.g., selected from 1, 2, 3, 4, 5, 6, 7, 8, 9, 10, 11, and 12), and b and c are independently integers from 1 to 5 (e.g., selected from 1, 2, 3, 4, and 5);
R* is a C₁₋₄ alkyl;
R^{o}, R″ and R′″ are each independently H or C₁₋₄ alkyl;
n is an integer of at least 5; and
A is a diol.

In a 10^{th} embodiment related to the composition of the first, second or third aspects above, and any one of embodiments 2-10, the delivery vehicle comprises a polyorthoester which has the structure of formula I where: R* is a methyl, ethyl, propyl or butyl, n is the number of repeating units and is an integer ranging from 5 to 400, and A in each subunit is R¹ or R³.

In an 11^{th} embodiment related to the composition of the first, second or third aspects above, and any one of embodiments 2-10, the delivery vehicle comprises a polyorthoester which has the structure of formula I where R* is ethyl.

In a 12^{th} embodiment related to the composition of the first, second or third aspects above, and any one of embodiments 2-10, the delivery vehicle comprises a polyorthoester which has the structure of formula I where: A corresponds to R¹, where R¹ is

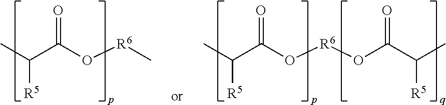

where p and q are each independently integers ranging from about 1 to 20, each R⁵ is independently hydrogen or C₁₋₄ alkyl; and R⁶ is:

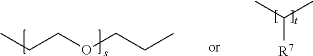

where s is an integer from 0 to 10; t is an integer from 2 to 30; and R⁷ is hydrogen or C₁₋₄ alkyl.

In a 13^{th} embodiment related to embodiment 12, R⁷ is H.

In a 14^{th} embodiment related to embodiment 12 or 13, the R¹ subunits are α-hydroxy acid-containing subunits.

In a 15^{th} embodiment related to any one of embodiments 12-14, p and q are each independently selected from 1, 2, 3, 4, 5, 6, 7, 8, 9, 10, 11, 12, 13, 14, 15, 16, 17, 18, 19, and 20.

In a 16^{th} embodiment related any one of embodiments 12-15, R⁵ is independently hydrogen, or C1, C2, C3, or C4 alkyl.

In a 17^{th} embodiment related to any one of embodiments 9-16, A corresponds to R³, where R³ is:

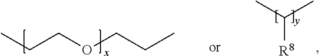

and x is an integer ranging from 1 to 100.

In an 18^{th} embodiment related embodiment 17, x is selected from 0, 1, 2, 3, 4, and 5; y is an integer in a range from 2 to 30; and R⁸ is hydrogen, a C₁₋₄ alkyl, a C1 alkyl, a C2 alkyl, a C3 alkyl, or a C4 alkyl.

In a 19^{th} embodiment related to the composition of the first or second aspects above, and any one of embodiments 1-18, the delivery vehicle comprises a polyorthoester which has the structure of formula I in which A is R¹ or R³, where R¹ is

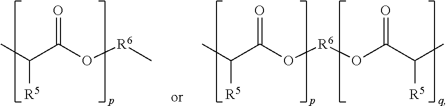

where p and q are each independently selected from 1, 2, 3, 4, 5, 6, 7, 8, 9, 10, 11, 12, 13, 14, 15, 16, 17, 18, 19, and 20 in any repeating unit, where the average number of p or the average number of the sum of p and q (p+q) is between about 1 and 7 or is 1, 2, 3, 4, 5, 6 or 7 in any repeating unit of R¹; x and s are each independently an integer ranging from 0 to 10; and t and y are each independently an integer ranging from 2 to 30, and R⁵ is H.

In a 20^{th} embodiment related to the composition of the first, second or third aspects above, and any one of embodiments 2-18, the delivery vehicle comprises a polyorthoester which has the structure of formula I in which A is R¹ or R³, where R¹ is

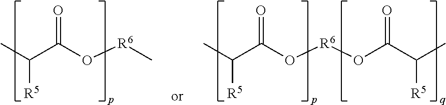

and p and q are each independently integers ranging from about 1 and 20, about 1 and 15, or about 1 and 10 in any repeating unit of R¹, where the average number of p or the average number of the sum of p and q is between about 1 and 7.

In a 21^{st} embodiment related to the composition of the first, second or third aspects above, and embodiment 20, x and s each independently range from 0 to about 7 or from 1 to about 5.

In a 22^{nd} embodiment related to the composition of the first, second or third aspects above, and embodiment 20 or 21, t and y each independently range from 2 to 10.

In a 23^{rd} embodiment related to the composition of the first, second or third aspects above, and any one of embodiments 20-22, R⁵ is hydrogen or methyl.

In a 24^{th} embodiment related to the composition of the first, second or third aspects above, and any one of embodiments 20-23, s and x are each independently selected from 1, 2, 3, 4, 5, 6, 7 and 8. In some particular embodiments, s is 2.

In a 25^{th} embodiment related to the composition of the first, second or third aspects above, and any one of embodiments 20-24, s is 2.

In a 26^{th} embodiment related to the composition of the first, second or third aspects above, and any one of embodiments 20-25, x is 2.

In a 21^{st} embodiment related to the composition of the first, second or third aspects above, and any one of embodiments 2-18, the delivery vehicle comprises a polyorthoester comprising alternating residues of 3,9-diethyl-3,9-2,4,8,10-tetraoxaspiro[5.5]undecane-3,9-diyl and A:

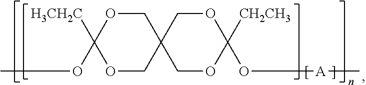

where A is as described in any one of embodiments 9-20.

In a 22^{nd} embodiment related to the composition of the first, second or third aspects above, and any one of embodiments 2-21, the delivery vehicle is a sustained release delivery vehicle which further comprises a solvent.

In a 23^{rd} embodiment, related to embodiment 22, the solvent is protic or aprotic in nature.

In a 24^{th} embodiment, related to embodiment 22 or 23, the solvent is dimethyl sulfoxide.

In a 25^{th} embodiment related to the composition of the first, second or third aspects above, and any one of embodiments 2-24, the delivery vehicle is a sustained release delivery vehicle and the active agents are solubilized in the sustained release delivery vehicle.

In a 26^{th} embodiment related to the composition of the first, second or third aspects above, and any one of embodiments 1-25, the delivery vehicle comprises a polyorthoester, olanzapine, granisetron, and DMSO.

In a 27^{th} embodiment related to embodiment 26, the combination of the polyorthoester and the solvent in the delivery vehicle is present in an amount ranging from about 85 to 98 wt %, 90 to 95 wt %, 93 to 95 wt %, or 93 to 97 wt %.

In a 28^{th} embodiment related to embodiments 26 and 27, the polyorthoester is present in the delivery vehicle in an amount ranging from about 65 to 95 wt %, 70 to 90 wt %, 70 to 85 wt %, 70 to 80 wt %, 75 to 90 wt %, 75 to 85 wt %, 80 to 90 wt %, or 85 to 90 wt %, or in an amount about 70 wt %, 73 wt %, 75 wt %, 78 wt %, 80 wt %, 85 wt % or 90 wt %.

In a 29^{th} embodiment related to embodiments 26-28, the solvent is present in the delivery vehicle in an amount ranging from about 5 to 30 wt %, 10 to 25 wt %, 10 to 23 wt %, 15 to 25 wt %, 15 to 20 wt %, 16 to 18 wt %, 20 to 25 wt %, or in an amount about 10 wt %, 15 wt %, 17 wt %, 20 wt %, 23 wt % or 25 wt %.

In a 30^{th} embodiment, related to the composition of the first, second or third aspects above, and any one of embodiments 1-29, the olanzapine is present the delivery vehicle in an amount ranging from about 1 to 8 percent, 1 to 6 percent, 1 to 5 percent, 0.5 to 5, 0.5 to 4 percent, 0.5 to 3 percent, 1 to 4 percent, 1 to 3 percent, or 1.5 to 2.5 percent by weight of the delivery system.

In a 31^{st} embodiment, related to the composition of the second or third aspects above, and any one of embodiments 1-30, the 5-HT3 receptor antagonist is present in the delivery vehicle in an amount ranging from about 1 to 10 percent, 1 to 8 percent, 1 to 6 percent, 1 to 5 percent, 0.5 to 5, 0.5 to 4 percent, 0.5 to 3 percent, 1 to 4 percent, 1 to 3 percent, or 1.5 to 2.5 percent by weight of the delivery system.

In a 32^{nd} embodiment, related to the composition of the second or third aspects above, and any one of embodiments 1-29, the olanzapine and the 5-HT3 receptor antagonist are present in the delivery vehicle in an amount ranging from about 0.5 to 8 percent and 1 to 10 percent, respectively; 0.5 to 5 percent and 1 to 10 percent, respectively; 1 to 6 percent and 1 to 8 percent, respectively; 1 to 5 percent and 2 to 6 percent, respectively; and 1 to 3 percent and 2 to 4 percent, respectively.

In a 33^{rd} embodiment, related to the composition of the second or third aspects above, and any one of embodiments 1-29, the 5-HT3 receptor antagonist is present in the delivery vehicle in an amount ranging from about 5 to 25 mg, 7 to 23 mg, 9 to 22 mg, 10 to 20 mg, 12 to 18 mg, 13 to 16 mg or about 12 mg, 14 mg, 15 mg, 16 mg, or 18 mg.

In a 31^{st} embodiment, related to the composition of the second or third aspects above, and any one of embodiments 1-29, the olanzapine and the 5-HT3 receptor antagonist are present in the delivery vehicle in an amount ranging from about 5 to 15 mg and 5 to 25 mg, respectively; 5 to 10 mg and 7 to 20 mg, respectively; 8 to 12 mg and 9 to 14 mg, respectively.

In a 32^{nd} embodiment, related to the composition of the second or third aspects above, and any one of embodiments 1-31, the delivery vehicle is a sustained release delivery vehicle and the olanzapine and the 5-HT3 receptor antagonist are released from the delivery vehicle over a period ranging from about 12 to 120 hours, about 24 to 120 hours, about 48 to 120 hours, about 72 to 120 hours, about 78 to 120 hours, about 84 to 120 hours, about 90 to 120 hours, about 96 to 120 hours, about 102 to 120 hours, about 108 to 120 hours, about 116 to 120 hours, about 48 to 96 hours, about 72 to 96 hours, about 78 to 96 hours, about 84 to 96 hours, about 72 to 132 hours, about 96 hours to 132 hours, or about 108 hours to 132 hours.

In a 33^{rd} embodiment, related to the composition of the first, second or third aspects above, and any one of embodiments 1-32, the 5-HT3 receptor antagonist is granisetron.

### EXAMPLES

The following examples are illustrative in nature and are in no way intended to be limiting.

### Example 1

### Granisetron and Olanzapine Delivery Systems

Compositions containing between 70% to 85% polyorthoester of formula I, between 10% and 23% of an aprotic, 3% to 5% olanzapine, and 2% granisetron. The solvents evaluated were dimethyl sulfoxide, n-methylpyrrolidone, and dimethylacetamide. Compositions of granisetron and olanzapine were prepared by dissolving 2% granisetron in the appropriate amount of solvent at 80° C. and 120° C. and then dissolving olanzapine in the heated solution of granisetron and solvent. The appropriate amount of POE was combined with the granisetron and olanzapine solution at an elevated temperature and mixed until homogenous. Granisetron and olanzapine compositions are presented in Table WW

TABLE 1-1

| | % | % | | |
|---|---|---|---|---|
| Formulation | Granisetron | Olanzapine | % DMSO | % POE |
| OG-01 | 2.0% | 5.0% | 20.0% | 73.0% |
| OG-02 | 2.0% | 5.0% | 23.0% | 70.0% |
| OG-03 | 2.0% | 3.0% | 10.0% | 85.0% |
| OG-04 | 2.0% | 3.0% | 15.0% | 80.0% |
| OG-05 | 2.0% | 3.0% | 17.0% | 78.0% |
| OG-06 | 2.0% | 3.0% | 20.0% | 75.0% |

### Example 2

### In-Vitro Release of Granisetron and Olanzapine Compositions

The release of granisetron and olanzapine from compositions generated as described in Example 1 was determined by placing a small amount of the polymer formulation (approximately 100 to 200 mg) into 150 mL of pH 6 phosphate buffered saline. The samples were then incubated at 50° C. with agitation. At 24 hour intervals, 1 mL samples were taken from the vials without agitation of the depot. Each sample was analyzed by HPLC to determine the concentration of granisetron and olanzapine. The cumulative drug release from the 100 mg or 200 mg depot was then calculated for granisetron and olanzapine; results are presented in Table 2-1 and Table 2-2, respectively.

TABLE 2-1
In-Vitro Release of Granisetron

| | Percent Granisetron Released for Compositions | | | | |
|---|---|---|---|---|---|
| Formulation | 0 | 1 | 2 | 3 | 4 |
| OG-02 | 0 | 35.10% | 71.00% | 103.80% | 106.50% |
| OG-04 | 0 | 21.00% | 40.70% | 86.80% | 99.60% |
| OG-05 | 0 | 25.60% | 44.20% | 85.10% | 102.60% |
| OG-06 | 0 | 19.90% | 25.80% | 67.10% | 100.10% |

TABLE 2-2
In-Vitro Release of Olanzapine

| | Percent Olanzapine Released for Compositions | | | | | |
|---|---|---|---|---|---|---|
| Formulation | 0 | 1 | 2 | 3 | 4 | 0 |
| OG-02 | 0 | 44.5% | 69.5% | 85.9% | 84.3% | 44.5% |
| OG-04 | 0 | 24.7% | 54.8% | 81.6% | 84.4% | 24.7% |
| OG-05 | 0 | 36.0% | 55.9% | 68.5% | 72.5% | 36.0% |
| OG-06 | 0 | 27.3% | 41.0% | 59.8% | 72.7% | 27.3% |

### Example 3

### Pharmacokinetic Analysis of Granisetron and Olanzapine Formulations in Canines

In a pharmacokinetic study, ten dogs (4 male-6 female) were treated with a formulation generated according to the method described in Example 1 and comprising 2.0 wt % granisetron, 3.0 wt % olanzapine, 15.0 wt % DMSO and 80.0 wt % polyorthoester. Dogs received the entire contents of 1 syringe containing sufficient polyorthoester formulation to deliver approximately 5 mg of granisetron and 7.5 mg of olanzapine. Plasma samples were taken from each dog at the following time points: 0, 0.5, 1, 3, 6, 8, 24, 48, 72, 96, 120 hours, and frozen. The plasma samples were subsequently analyzed by LC/MS/MS for granisetron and olanzapine. A plot of the plasma concentration of granisetron and olanzapine versus time is presented in FIG. 3. The formulations provided measurable plasma concentrations of granisetron and olanzapine for 5 days. All formulations provided measurable plasma concentrations of granisetron and olanzapine for at least 5 days.

TABLE 3-1
Average Plasma Concentrations of Granisetron

| Time (hrs) | Granisetron (ng/ml) | Std Dev |
|---|---|---|
| 0 | 0 | 0 |
| 0.5 | 11.71 | 3.88 |
| 1 | 14.35 | 4.29 |
| 3 | 14.17 | 5.47 |
| 6 | 10.66 | 7.07 |
| 8 | 8.02 | 4.10 |
| 24 | 6.82 | 3.36 |
| 48 | 3.27 | 1.18 |
| 72 | 1.54 | 0.50 |
| 96 | 0.95 | 0.38 |
| 120 | 0.40 | 0.35 |

TABLE 3-2
Average Plasma Concentrations of Olanzapine

| Time (hrs) | Olanzapine (ng/ml) | Std Dev |
|---|---|---|
| 0 | 0 | 0 |
| 0.5 | 1.53 | 0.56 |
| 1 | 1.87 | 0.90 |
| 3 | 2.40 | 0.81 |
| 6 | 2.71 | 0.89 |
| 8 | 2.90 | 0.73 |
| 24 | 2.62 | 0.88 |
| 48 | 1.83 | 0.42 |
| 72 | 1.65 | 0.46 |
| 96 | 1.24 | 0.42 |
| 120 | 0.96 | 0.35 |

It is claimed:

1. A pharmaceutical composition, comprising: olanzapine, a 5-HT3 receptor antagonist and a delivery vehicle.

2. The composition of claim 1, wherein the 5-HT3 receptor antagonist is selected from the group consisting of granisetron, tropisetron, ondansetron, palonosetron, and dolasetron.

3. The composition of claim 1, wherein the 5-HT3 receptor antagonist is granisetron.

4. The composition of claim 1, wherein the olanzapine is present in the composition in an amount between about 1 wt % and about 5 wt %.

5. The composition of claim 1, wherein the 5-HT3 receptor antagonist is present in an amount between about 2 wt % and about 8 wt %.

6. The composition of claim 1, wherein the delivery vehicle is a sustained-release delivery vehicle.

7. The composition of claim 6, wherein the sustained-release delivery vehicle is a polymeric formulation, a liposome, a microsphere, an implantable device or a non-polymeric formulation.

8. The composition of claim 6, wherein the sustained-release delivery vehicle is a liposome selected from the group consisting of small unilamellar vesicles (SUV), large unilamellar vesicles (LUV), multi-lamellar vesicles (MLV) and multivesicular liposomes (MVL).

9. The composition of claim 8, wherein the olanzapine and the 5-HT3 receptor antagonist are entrapped in an aqueous space of the liposome or in a lipid layer of the liposome.

10. The composition of claim 6, wherein the sustained-release delivery vehicle is a microsphere comprised of a bioerodible or biodegradable polymer.

11. The composition of claim 10, wherein the olanzapine and the 5-HT3 receptor antagonist are entrapped in the microsphere.

12. The composition of claim 6, wherein the sustained-release delivery vehicle is an osmotic pump with a reservoir comprising the olanzapine and 5-HT3 receptor antagonist.

13. The composition of claim 6, wherein the sustained-release delivery vehicle is a non-polymeric formulation comprising sucrose acetate isobutyrate.

14. The composition of claim 6, wherein the sustained-release delivery vehicle is a polymeric formulation in the form of a semi-solid polymer formulation comprising a polymer, the olanzapine and the 5-HT3 receptor antagonist.

15. The composition of claim 14, wherein the polymer is a bioerodible or biodegradable polymer.

16. The composition of claim 15, wherein the polymer formulation forms an implant or depot in situ.

17. The composition of claim 15, wherein the polymer is selected from the group consisting of polylactides, polyglycolides, poly(lactic-co-glycolic acid) copolymers, polycaprolactones, poly-3-hydroxybutyrates, and polyorthoesters.

18. The composition of claim 15, wherein the polymer is a polyorthoester.

19. The composition of claim 18, wherein the polyorthoester is selected from the polyorthoesters represented by Formulas I, II, III and IV.

20. The composition of claim 18, wherein the polyorthoester is represented by Formula I.

21. The composition of claim 20, wherein the composition has a viscosity ranging from about 2500 mPa-s to 10000 mPa-s when measured at 25° C. or 37° C. using a viscometer.

22. The composition of claim 6, wherein the olanzapine and the 5-HT3 receptor antagonist are released from the composition over a time period of about 1 day to about 8 weeks.

23. A method for treating chemotherapy induced nausea and vomiting (CINV) in a subject in need thereof, comprising administering to the subject a composition according to claim 1.

24. A method for prophylactic treatment of CINV in a subject in need thereof, comprising: administering to the subject a composition according to claim 1.

25. A pharmaceutical composition, comprising: olanzapine and a delivery vehicle.

26. The composition of claim 25, wherein the olanzapine is present in the composition in an amount between about 1 wt % and about 20 wt %.

27. The composition of claim 25, wherein the olanzapine is present in the composition in an amount between about 5 wt % and about 10 wt %.

28. The composition of claim 25, wherein the delivery vehicle is a sustained-release delivery vehicle.

29. A method for treating chemotherapy induced nausea and vomiting (CINV) in a subject in need thereof, comprising administering to the subject a composition according to claim 25.

30. A method for prophylactic treatment of CINV in a subject in need thereof, comprising: administering to the subject a composition according to claim 25.
